(19) United States
(12) Patent Application Publication
(10) Pub. No.: US 2010/0035929 A1
(43) Pub. Date: Feb. 11, 2010

(54) UNNATURAL DISPYRIN ANALOGUES, PREPARATION AND USES THEREOF

(76) Inventors: Craig W. Lindsley, Brentwood, TN (US); P. Jeffrey Conn, Brentwood, TN (US); J. Phillip Kennedy, Nashville, TN (US); John T. Brogan, San Marino, CA (US)

Correspondence Address: Ballard Spahr LLP, SUITE 1000, 999 PEACHTREE STREET, ATLANTA, GA, 30309-3915, US

(21) Appl. No.: 12/480,069

(22) Filed: Jun. 8, 2009

Related U.S. Application Data
(60) Provisional application No. 61/059,975, filed on Jun. 9, 2008.

(51) Int. Cl. A61K 31/40; C07D 401/12; C07D 413/12; C07D 417/12; C07D 403/12; C07D 207/09; C07D 409/12; A61K 31/4439; A61K 31/422; A61K 31/4196; A61K 31/427; A61K 31/4025; A61K 31/4535; A61K 31/4525; A61K 31/454; A61P 9/00; A61P 9/12; A61P 3/10; A61P 1/00; A61P 19/02; A61P 35/00; A61P 25/28; A61P 25/06; A61P 25/00

(52) U.S. Cl. 514/326; 546/276.4; 548/236; 548/200; 548/266.8; 548/575; 548/527; 546/213; 514/343; 514/374; 514/383; 514/365; 514/428; 514/422

(57) ABSTRACT

Disclosed are dispyrin analogue compounds useful as H3 receptor activity modulators, methods of making same, pharmaceutical compositions comprising same, and methods of treating neurological and psychiatric disorders associated with histamine H3 receptor activity using same. In one aspect, the disclosed analogues can have a structure represented by a formula:

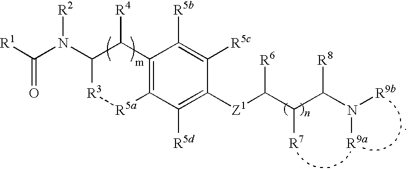

This abstract is intended as a scanning tool for purposes of searching in the particular art and is not intended to be limiting of the present invention.

### CROSS-REFERENCE TO RELATED APPLICATIONS

This application claims the benefit of U.S. Application No. 61/059,975, filed Jun. 9, 2008, which is hereby incorporated herein by reference in its entirety.

### BACKGROUND

Monoamines, including histamine, can act as multifunctional chemical transmitters that signal through cell surface receptors linked to intracellular pathways via guanine nucleotide binding proteins. Such cell surface receptors are called G-protein coupled receptors or GPCRs. Currently, there are three subtypes of histamine receptors that have been defined pharmacologically and have been divided into H1, H2, and H3 classifications, with a newly identified receptor designated GPRv53 [(Oda T., et al., J. Biol. Chem. 275 (47): 36781-6 (2000)]. The H1 histamine receptor has been cloned and is the target of drugs such as diphenhydramine to block the effects of histamine during allergic responses. The H2 histamine receptor has also been cloned and is the target of drugs such as ranitidine to block the effects of histamine on acid secretion in the stomach. The third subtype, H3, is believed to function as a presynaptic autoreceptor in histamine containing neurons in the central nervous system and as a presynaptic heteroreceptor in non-histamine containing neurons. One of the functions of the H3 receptor is to regulate neurotransmitter release at a presynaptic site. Histamine H3 receptors are thus expressed in the central nervous system, but have also been pharmacologically identified in heart, lung, and gastrointestinal tract, and have been hypothesized to exist in other tissues.

The histamine H3 receptor is relatively neuron specific and inhibits the release of a number of monoamines, including histamine. Recent evidence suggests that the H3 receptor shows intrinsic, constitutive activity, in vitro as well as in vivo (i.e., it is active in the absence of an agonist; see e.g., Morisset et al., Nature 2000, 408, 860-864). Compounds acting as inverse agonists or antagonists can inhibit this activity. The histamine H3 receptor has been demonstrated to regulate the release of histamine and also of other neurotransmitters such as serotonin and acetylcholine. A histamine H3 receptor antagonist or inverse agonist would therefore be expected to increase the release of these neurotransmitters in the brain. By increasing the release of neurotransmitters in the brain, an H3 receptor antagonist or inverse agonist can inhibit activities such as food consumption while minimizing non-specific peripheral consequences. Antagonists or inverse agonists of the histamine H3 receptor can also increase synthesis and release of cerebral histamine and other monoamines. By this mechanism, they induce a prolonged wakefulness, improved cognitive function, and normalization of vestibular reflexes. Accordingly, the histamine H3 receptor is an important target for new therapeutics for Alzheimer disease, mood and attention adjustments, cognitive deficiencies, obesity, dizziness, schizophrenia, epilepsy, sleeping disorders, narcolepsy and motion sickness, neuropathic pain, among others.

The majority of histamine H3 receptor antagonists resemble histamine in possessing an imidazole ring generally substituted in the 4(5) position (Ganellin et al., Ars Pharmaceutica, 1995, 36:3, 455-468). These imidazole-containing compounds have the disadvantage of poor blood-brain barrier penetration, interaction with cytochrome P-450 proteins, and hepatic and ocular toxicities. Non-imidazole neuroactive compounds such as beta histamines (Arrang, Eur. J. Pharm. 1985, 111:72-84) show some histamine H3 receptor activity but with poor potency.

Therefore, there remains a need for compounds and compositions useful as histamine H3 receptor modulators that overcome current deficiencies and that effectively treat diseases and disorders associated with histamine H3 receptor activity.

### SUMMARY

In accordance with the purpose(s) of the invention, as embodied and broadly described herein, the invention, in one aspect, relates to unnatural dispyrin analogue compounds useful as H3 receptor activity modulators, methods of making same, pharmaceutical compositions comprising same, and methods of treating neurological and psychiatric disorders associated with histamine H3 receptor activity using same.

Disclosed are compounds comprising a structure represented by a formula:

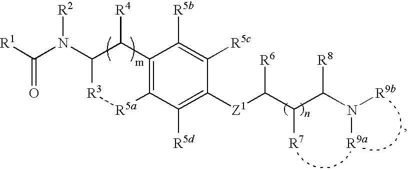

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof, with the proviso that the compound is not Dispyrin, Purealidin Q, Purealidin S, Purpurealidin A, Purpurealidin B, or Fistularin-3.

Also disclosed are synthetic compounds comprising a structure represented by a formula:

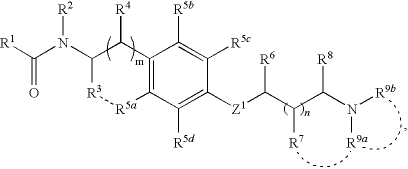

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof.

Also disclosed are methods of preparing a compound comprising the step of reacting a compound comprising a structure represented by a formula:

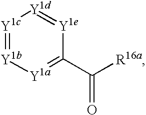

wherein each of Y^{1a}, Y^{1b}, Y^{1c}, Y^{1d}, and Y^{1e} is independently selected from nitrogen or CR¹¹, wherein each R¹¹, when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons; wherein R^{16a} comprises OH, alkoxy, acyloxy, hydrogen, or halogen; with the proviso that no more than two of Y^{1a}, Y^{1b}, Y^{1c}, Y^{1d}, and Y^{1e} are nitrogen; or

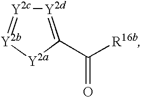

wherein Y^{2a} is selected from O, S, and NR¹², wherein R¹², if present, is selected from hydrogen or an alkyl residue comprising from 1 to 4 carbons; wherein each of Y^{2b}, Y^{2c}, and Y^{2d} is independently selected from N and CR¹², wherein each R¹², when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons; wherein R^{16b} comprises OH, alkoxy, acyloxy, hydrogen, or halogen; with the proviso that no more than three of Y^{2a}, Y^{2b}, Y^{2c}, and Y^{2d} are heteroatoms, with a compound having a structure represented by a formula:

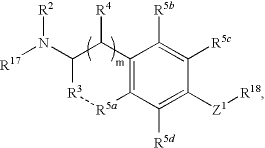

wherein R² is selected from hydrogen, an optionally substituted organic residue comprising from 1 to 6 carbons, or a hydrolysable residue; wherein each of R³, and R⁴ (if present) independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein ---- is an optional covalent bond; wherein m an integers selected from 0, 1, and 2; wherein each of R^{5a}, R^{5b}, R^{5c} and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; wherein R¹⁷ is hydrogen or a hydrolyzable group; wherein R¹⁸ is hydrogen, a hydrolyzable group, a protecting group, or an optionally substituted organic residue comprising from 1 to 12 carbons; thereby forming an amide bond.

Also disclosed are the products of the disclosed methods.

Also disclosed are methods of modulating the activity of a G-protein coupled receptor in at least one cell comprising the step of contacting the at least one cell with at least one compound comprising a structure represented by a formula:

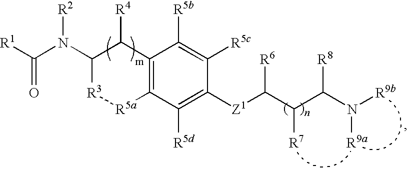

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof, thereby modulating activity of the G-protein coupled receptor in the at least one cell.

Also disclosed are methods of modulating the activity of a G-protein coupled receptor in a subject in need thereof comprising the step of administering to the subject a therapeutically effective amount of at least one compound comprising a structure represented by a formula:

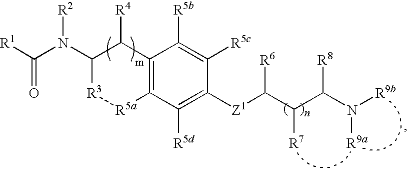

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof, thereby modulating activity of the G-protein coupled receptor in the subject.

Also disclosed are methods for treating a disorder associated with G-protein coupled receptor activity in a subject comprising the step of administering to the subject a therapeutically effective amount of at least one compound comprising a structure represented by a formula:

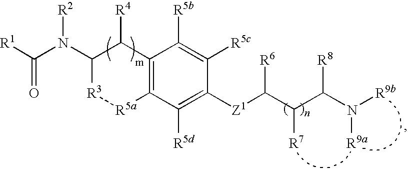

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof, thereby treating the disorder in the subject.

Also disclosed are dosage forms comprising at least one compound comprising a structure represented by a formula:

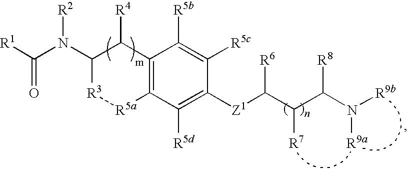

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof, and a pharmaceutically acceptable carrier.

### BRIEF DESCRIPTION OF THE FIGURES

The accompanying figures, which are incorporated in and constitute a part of this specification, illustrate several aspects and together with the description serve to explain the principles of the invention.

FIG. 1 shows a class of exemplary disclosed dispyrin analogues.

FIG. 2 shows the oroidin class of bromopyrrole carboxamide alkaloids from Agelas.

FIG. 3 shows exemplary bromotyrosine alkaloids isolated from marine sources.

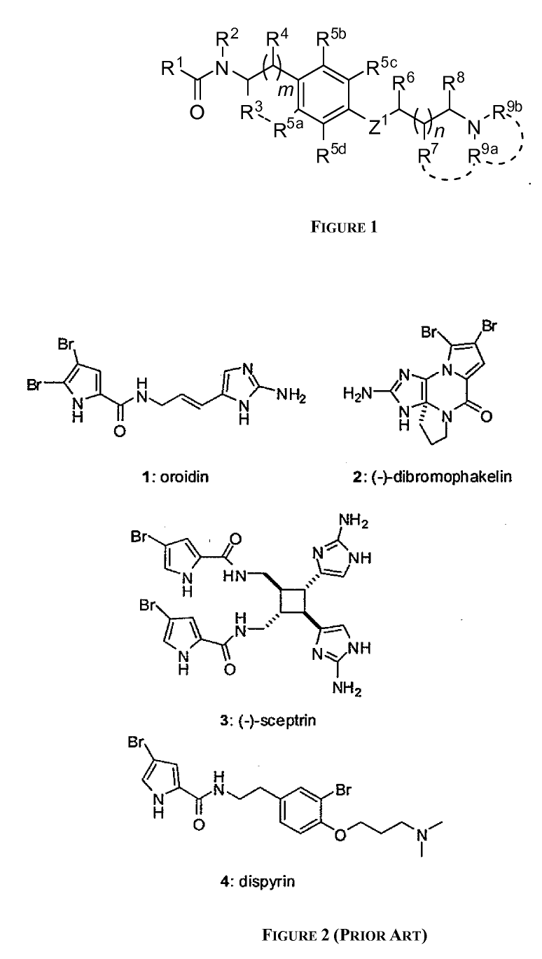

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which may need to be independently confirmed.

### A. DEFINITIONS

As used in the specification and the appended claims, the singular forms “a,” “an” and “the” include plural referents unless the context clearly dictates otherwise. Thus, for example, reference to “a component,” “a polymer,” or “a particle” includes mixtures of two or more such components, polymers, or particles, and the like.

Ranges can be expressed herein as from “about” one particular value, and/or to “about” a further particular value. When such a range is expressed, a further embodiment includes from the one particular value and/or to the other particular value. Similarly, when values are expressed as approximations, by use of the antecedent “about,” it will be understood that the particular value forms a further embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance may or may not occur, and that the description includes instances where said event or circumstance occurs and instances where it does not.

As an example, the term “optionally substituted,” as used herein, means that the groups in question are either unsubstituted or substituted with one or more of the substituents specified. When the groups in question are substituted with more than one substituent the substituents can be the same or different.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinbelow. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

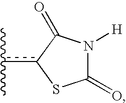

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

The term “alkyl” as used herein is a branched or unbranched saturated hydrocarbon group of 1 to 24 carbon atoms, e.g. 1 to 18 carbons atoms, 1 to 14 carbon atoms, 1 to 12 carbon atoms, 1 to 10 carbon atoms, 1 to 8, 1 to 6 carbon atoms, or 1 to 4 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, halide, hydroxamate, hydroxy, ketone, nitro, silyl, sulfo-oxo, sulfonyl, sulfone, sulfoxide, or thiol, as described below. The term “halogenated alkyl” specifically refers to an alkyl group that is substituted with one or more halide, e.g., fluorine, chlorine, bromine, or iodine. A “lower alkyl” group is an alkyl group containing from one to six (e.g., from one to four) carbon atoms.

The term “alkoxy” as used herein is an alkyl group bound through a single, terminal ether linkage; that is, an “alkoxy” group may be defined as —OA where A is alkyl as defined above.

The term “alkenyl” as used herein is a hydrocarbon group of from 2 to 24 carbon atoms with a structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C═C.

The term “alkynyl” as used herein is a hydrocarbon group of 2 to 24 carbon atoms with a structural formula containing at least one carbon-carbon triple bond, i.e., C≡C.

The term “aryl” as used herein is any carbon-based aromatic group including, but not limited to, benzene, naphthalene, phenyl, biphenyl, etc. The term “aromatic” also includes “heteroaryl,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, halide, hydroxamate, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, halide, hydroxamate, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and contains at least one carbon-carbon double bound, C═C. Examples of cycloalkenyl groups include, but are not limited to, cyclopropenyl, cyclobutenyl, cyclopentenyl, cyclopentadienyl, cyclohexenyl, etc. The term “heterocycloalkenyl” is a cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, halide, hydroxamate, hydroxy, ketone, nitro, silyl, sulfo-oxo, sulfonyl, sulfone, sulfoxide, or thiol as described herein.

The term “heterocycle” as used herein is intended to include the following groups, which can be optionally substituted: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof.

The term “heterocycle” as used herein is also intended to include, but is not limited to, the following groups which can be optionally substituted: methylenedioxyphenyl, imidazopyridyl, imidazopyrimidinyl, imidazopyridazinyl, imidazopyrazinyl, imidazotriazinyl, imidazothiopheyl, pyrazolopyridyl, pyrazolopyrimidinyl, pyrazolopyridazinyl, pyrazolopyrazinyl, pyrazolotriazinyl, pyrazolothiophenyl, triazolopyridyl, triazolopyrimidinyl, triazolopyridazinyl, triazolopyrazinyl, triazolothiophenyl, tetrahydroimidazopyridinyl, tetrahydropyrazolopyridinyl, tetrahydrotriazopyridinyl, tetrahydrotriazolopyridazinyl, and tetrahydroindazolyl.

The term “heterocycle” as used herein is also intended to include, but is not limited to, the following groups which can be optionally substituted: tetrahydroimidazopyrimidyl, tetrahydroimidazopyrazinyl, tetrahydroimidazopyridazinyl, tetrahydrotriazolopyrimidyl, tetrahydrotriazolopyrazinyl, tetrahydropyrazolopyrimidyl, tetrahydropyrazolopyrazinyl, imidazothiazolyl, and imidazothiadiazolyl.

The term “aldehyde” as used herein is represented by the formula —C(O)H.

The terms “amine” or “amino” as used herein are represented by the formula —NAA¹A², where A, A¹, and A² can be, independently, any suitable substituent, including hydrogen, alkyl, halogenated alkyl, alkenyl, alkynyl, aryl, heteroaryl, cycloalkyl, cycloalkenyl, heterocycloalkyl, or heteroalkenyl group described above. An amino group can be present as an N-oxide. An “N-oxide,” as used herein is represented by a formula N(O)AA¹A², where A, A¹, and A² are as defined above. An “N-oxide” can comprise a dative bond, i.e., N→O, which is sometimes represented by the formula, N═O.

The term “carboxylic acid” as used herein is represented by the formula —C(O)OH.

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula AOA1, where A and A1 can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula —C(O)—.

The term “halide” as used herein refers to the halogens fluorine, chlorine, bromine, and iodine.

The term “hydroxamate” as used herein is represented by the formula—C(O)NHOH.

The term “hydroxyl” as used herein is represented by the formula —OH.

The term “nitro” as used herein is represented by the formula —NO₂.

The term “thiol” as used herein is represented by the formula —SH.

The term “cyano” as used herein is represented by the formula —CN.

The term “azide” as used herein is represented by the formula —N₃.

The term “carboxamido” as used herein is represented by the formula —C(O)NH—.

The term “trifluoromethyl” as used herein is represented by the formula —CF₃.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixtures.

As used herein, and without limitation, the term “derivative” is used to refer to any compound which has a structure derived from the structure of the compounds disclosed herein and whose structure is sufficiently similar to those disclosed herein and based upon that similarity, would be expected, by one skilled in the art, to exhibit the same or similar activities and utilities as the claimed compounds, or to induce, as a precursor, the same or similar activities and utilities as the claimed compounds.

The term “pharmaceutically acceptable” means a material that is not biologically or otherwise undesirable, i.e., without causing any undesirable biological effects or interacting in a deleterious manner.

The term “pharmaceutically acceptable derivative” refers to any homolog, analog, or fragment corresponding to the disclosed compounds which can modulate spliceosome activity. A “pharmaceutically acceptable derivative,” for example, includes any pharmaceutically acceptable salt, ester, amide, salt of an ester or amide, or other derivative of a disclosed compound.

The term “hydrolysable residue” is meant to refer to a functional group capable of undergoing hydrolysis, e.g., under basic or acidic conditions. Examples of hydrolysable residues include, without limitatation, acid halides, activated carboxylic acids, and various protecting groups known in the art (see, for example, “Protective Groups in Organic Synthesis”, T. W. Greene, P. G. M. Wuts, Wiley-Interscience, 1999).

The term “leaving group” refers to an atom (or a group of atoms) with electron withdrawing ability that can be displaced as a stable species, taking with it the bonding electrons. Examples of suitable leaving groups include sulfonate esters, including, but not limited to, triflate, mesylate, tosylate, brosylate, and halides.

Certain instances of the above defined terms may occur more than once in the structural formulae, and upon such occurrence each term shall be defined independently of the other.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound If given a name, either by systemic reduction of the compound structure using naming conventions, or by commercially available software, such as CHEMDRAW™ (Cambridgesoft Corporation, U.S.A.).

As used herein, a “G-protein coupled receptor” is meant to refer to a transmembrane (cell membrane) receptor protein that can sense molecules outside the cell and activate signal transduction pathways inside the cell, and, ultimately, cellular responses. An example of a G-protein couple receptor is a histamine H3 receptor, which senses, among others, histamine, and ultimately modulates neurocellular responses.

The term “modulate” or “modulating” refers to the ability of an agent (e.g., an H3 modulator) to regulate a desired response, e.g. H3 activity. Modulate, as used herein, can refer to a process by which an agent elevates or reduces a desired response. Modulate refers to the ability of an agent to regulate a response either directly or indirectly. Modulate can refer to a process by which an agent substantially inhibits, stabilizes, or prevents a response when a response would otherwise increase. Modulate can also refer to a process by which an agent substantially stabilizes, enhances, or maintains a response when an immune response would otherwise decrease. Thus, compounds disclosed herein as H3 modulators, can function as inhibitory agents, inverse agonists, or antagonists, for example. Included within “inhibitory agents” is a preventative agent, i.e. a compound capable of an H3 blockade or shutdown.

The term “H3” refers to the histamine H3 receptor that regulates, including inhibits, the release of a number of monoamines, including histamine.

As used herein, the term “H3 modulator” refers to any exogenously administered compound or agent that directly elevates or reduces (increases or decreases) the activity of the histamine H3 receptor in an animal, in particular a mammal, for example a human. This term includes “H3 agonists” and “H3 antagonists.”

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein.

The term “contacting” as used herein refers to bringing a disclosed compound and a cell, target histamine receptor, or other biological entity together in such a manner that the compound can affect the activity of the target (e.g., spliceosome, cell, etc.), either directly; i.e., by interacting with the target itself, or indirectly; i.e., by interacting with another molecule, co-factor, factor, or protein on which the activity of the target is dependent.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

By “treatment,” it is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement a further specific therapy directed toward the improvement of the associated disease, pathological condition, or disorder. The term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

By the term “therapeutically effective amount” of a compound or composition as provided herein is meant a nontoxic but sufficient amount of the compound to provide the desired utility, for example to reduce, inhibit, prevent, or otherwise modulate a desired response. As will be pointed out below, the exact amount required will vary from subject to subject, depending on the species, age, body weight, general health, sex, diet, and general condition of the subject, the severity of the condition or disease that is being treated, the particular compound used, its mode of administration, the duration of the treatment; drugs used in combination or coincidental with the specific composition employed, and like factors well known in the medical arts. Thus, it is not possible to specify an exact “effective amount”; however, an appropriate effective amount can be determined by one of ordinary skill in the art using only routine experimentation. For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for the treatment of a disease. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: 1) a subject's physical condition is shown to be improved, 2) the progression of the disease or condition is shown to be stabilized, or slowed, or reversed, or 3) the need for other medications for treating the disease or condition is lessened or obviated, then a particular treatment regimen will be considered efficacious. If desired, the effective daily dose can be divided into multiple doses for purposes of administration. Consequently, single dose compositions can contain such amounts or submultiples thereof to make up the daily dose. The dosage can be adjusted by the individual physician or the subject in the event of any counterindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

By “prevent” or “preventing” is meant to preclude, avert, obviate, forestall, stop, or hinder something from happening, especially by advance action. It is understood that where reduce, inhibit or prevent are used herein, unless specifically indicated otherwise, the use of the other two words is also expressly disclosed.

As used herein, “diagnosed” means having been subjected to a physical examination by a person of skill, for example, a physician, and found to have a condition that can be diagnosed or treated by the compounds, compositions, or methods disclosed herein. As one example, “diagnosed” can refer to having been subjected to a physical examination by a person of skill, for example, a physician, and found to have a condition that can be diagnosed or treated by a compound or composition that can modulate (increase or decrease) H3 receptor activity.

Disclosed are the components to be used to prepare the compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions. Thus, if there are a variety of additional steps that can be performed it is understood that each of these additional steps can be performed with any specific embodiment or combination of embodiments of the methods.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. DISPYRIN ANALOGUES

In one aspect, the present disclosure relates to disyrin, a newly discovered marine natural product, and synthetic analogues related thereto. Dispyrin belongs to a class of bromopyrrole carboxamide alkloids known as the oriodin class, derived from the genus Agelas. Sponges of the genus Agelas, found throughout the world's tropical reefs, have provided a variety of bromopyrrole carboxamide containing alkaloids derived biosynthetically from oroidin 1 (FIG. 2). Examples include the teratcyclic alkaloid (−)-dibromophakelin 2 and the tetrasubstituted cylobutane marine alkaloid (−)-sceptrin 3 (FIG. 2).

Recently, Crews and co-workers reported a new study related to the Carribean sponge Agelas dispar. [I. C. Pina, K. N. White, G. Cabrera, E. Rivero, P. Crews, J. Nat. Prod. 2007, 70, 613-617.] The Crews effort was focused on discovering compounds with interesting molecular architectures. Dispyrin 4 (FIG. 2) contains a bromopyrrole tyramine motif that has no precedent in marine natural products research. Moreover, unlike all bromopyrrole carboxamide alkaloids discovered from Agelas thus far, dispyrin is not biosynthetically derived from oroidin 1, but rather an independent biosynthetic pathway. The Crews study did not elucidate any biological activity for dispyrin 4.

In one aspect, the present disclosure relates to a pharmacore analysis of dispyrin and analogues related thereto, and the discovery of the biological activity of dispyrin and dispyrin analogues. It was recognized that dispyrin possesses a topology and pharamacophoric elements reminiscent of therapeutically relevant ligands for GPCRs (G Protein-Coupled Receptors) and inhibitors of various ion channels. Thus, a program to synthesize dispyrin 4 and elucidate the molecular target(s) of this newly discovered bromopyrrole carboxamide alkaloid was initiated.

An exemplary retrosynthetic analysis is represented in Scheme 1, which can allow for the synthesis of a diverse library of dispyrin analogous, including a synthetic dispyrin. It should be appreciated that such a library can serve to establish structure-activity-relationships (SAR). For the library, chemical yields for each step averaged in the 80-95% range with overall yields in the 50+% range. Each member of the library was purified by mass-directed preparative HPLC to analytical purity and fully characterized.

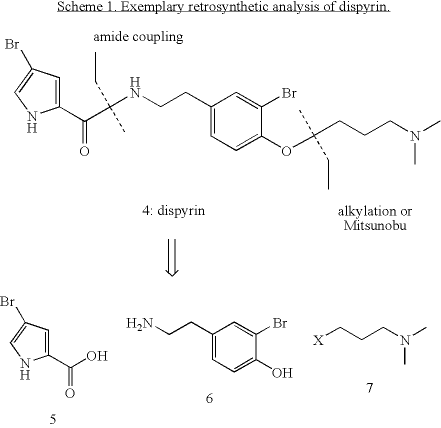

It should be appreciated that synthetic dispyrin was found to provide inhibition (50-60% at 10 μm) of a number of calcium and potassium ion channels, including hERG. It was also found that dispyrin possesses affinity for the human H3 receptor (Kᵢ=1.04 μm in a radioligand binding assay employing [³H](R-α-methylhistamine) and was a moderately potent human H3 antagonist (IC₅₀=2.35 μm).

### B. COMPOUNDS

1. Structure

In one aspect, disclosed herein are compounds or pharmaceutically acceptable derivatives thereof which have selective histamine-H3 receptor antagonist activity, inverse agonist activity, or inhibitory activity, as well as methods for preparing such compounds. The compounds disclosed herein can be useful in the treatment neurological and psychiatric disorders, among others, associated with H3 receptor activity, as further described herein.

In one aspect, disclosed herein are compounds comprising a structure represented by a formula:

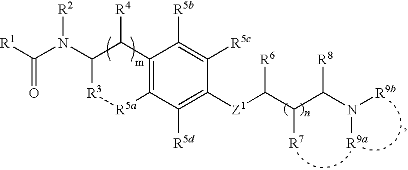

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof, with the proviso that the compound is not Dispyrin, Purealidin Q, Purealidin S, Purpurealidin A, Purpurealidin B, or Fistularin-3.

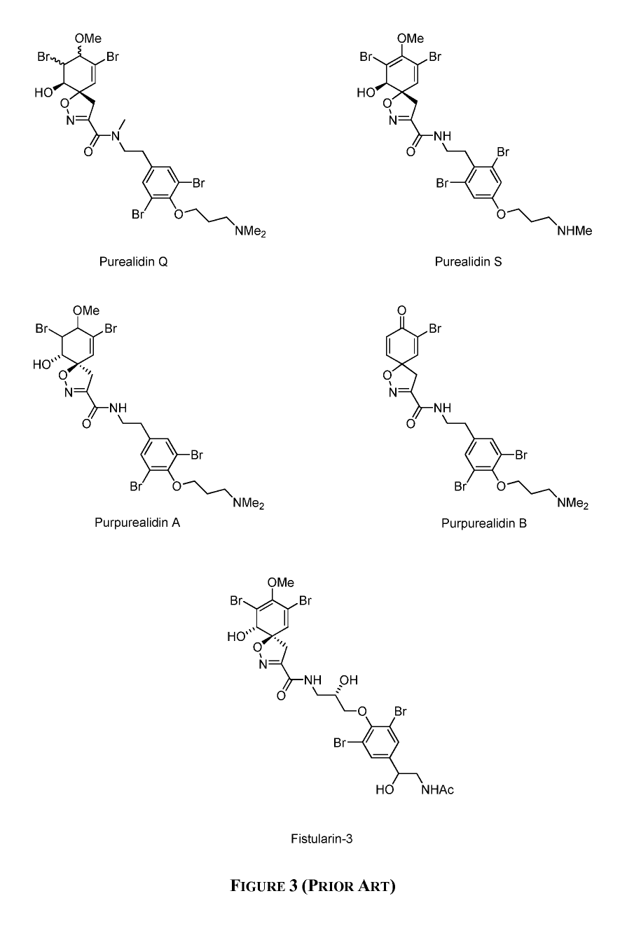

In one aspect, the compound is not a bromotyrosine alkaloid isolated from nature, such as, for example, Dispyrin, Purealidin Q, Purealidin S, Purpurealidin A, Purpurealidin B, or Fistularin-3 (FIG. 3). In a further aspect, the compound can be a synthetic bromotyrosine alkaloid with a structure corresponding to that of a natural analogue.

In a further aspect, disclosed herein are synthetic compounds or pharmaceutically acceptable derivatives including, for example, synthetic Dispyrin.

In one aspect, R¹ comprises a structure represented by a formula:

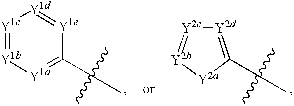

wherein each of Y^{1a}, Y^{1b}, Y^{1c}, Y^{1d}, and Y^{1e} is independently selected from nitrogen or CR¹¹, wherein each R¹¹, when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons; with the proviso that no more than two of Y^{1a}, Y^{1b}, Y^{1c}, Y^{1d}, and Y^{1e} are nitrogen; and wherein Y^{2a} is selected from O, S, and NR¹², wherein R¹², if present, is selected from hydrogen or an alkyl residue comprising from 1 to 4 carbons; wherein each of Y^{2b}, Y^{2c}, and Y^{2d} is independently selected from N and CR¹², wherein each R¹², when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons; with the proviso that no more than three of Y^{2a}, Y^{2b}, Y^{2c}, and Y^{2d} are heteroatoms.

In a further aspect, R¹ is selected from optionally substituted pyridyl, optionally substituted oxazolyl, optionally substituted triazolyl, optionally substituted thiazolyl, optionally substituted aryl, optionally substituted thiopheneyl, optionally substituted pyrrolyl, optionally substituted alkyl pyrrolyl, and optionally substituted furanyl.

In a still further aspect, R¹ is selected from:

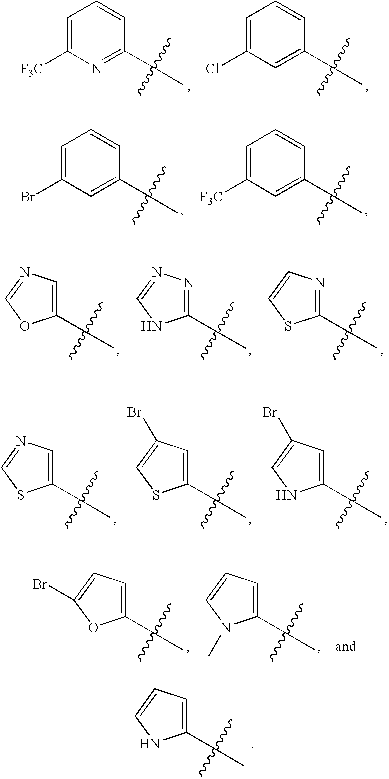

In one aspect, R² can be hydrogen. Likewise, in a further aspect, each of R³ and R⁴, when present, comprises two hydrogens.

In one aspect, m is 1.

In a further aspect, at least one of R^{5a}, R^{5b}, R^{5c}, and R^{5d} is halogen.

In a further aspect, at least one of R^{5c} and R^{5d} is halogen.

In one aspect, each of R^{5a} and R^{5b} independently comprises hydrogen, and one of R^{5c} and R^{5d} is halogen, and one of R^{5c} and R^{5d} is hydrogen.

In a further aspect, Z¹ is oxygen.

In a further aspect, R⁶ comprises two hydrogens.

In one aspect, n is 0. In a further aspect, n is 1.

In one aspect, R⁷ comprises two hydrogens.

In a further aspect, R⁸ comprises two hydrogens.

In a further aspect, each of R^{9a} and R^{9b} comprises methyl.

In a further aspect, R^{9a}, N, and R^{9b} together comprise an optionally substituted heterocycle comprising from 2 to 12 carbons.

In one aspect, the compound comprises a structure represented by the formula:

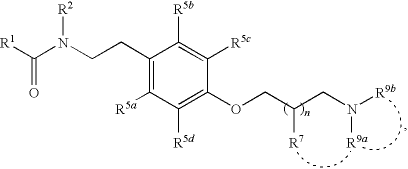

wherein at least one of R^{5c} and R^{5d} is halogen.

In a further aspect, the compound comprises a structure represented by a formula:

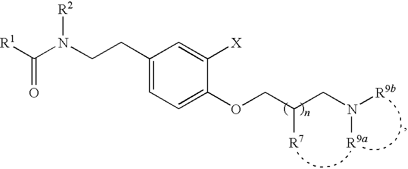

wherein X is F, Cl, Br, or I; and wherein n is an integer from 0 to 1.

In a still further aspect, the compound comprises a structure represented by a formula selected from:

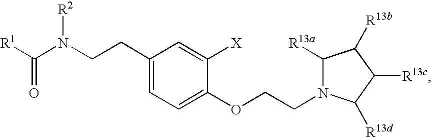

wherein X is F, Cl, Br, or I; and wherein each of R^{13a}, R^{13b}, R^{13c}, and R^{13d} is independently selected from hydrogen, alkyl comprising from 1 to 4 carbons, and halide; or

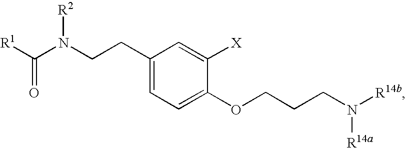

wherein X is F, Cl, Br, or I; and wherein each of R^{14a} and R^{14b} is independently selected from hydrogen and alkyl comprising from 1 to 4 carbons; or

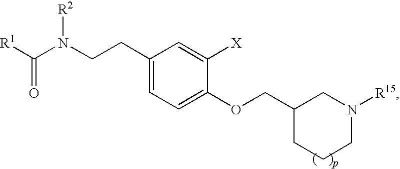

wherein X is F, Cl, Br, or I; wherein R¹⁵ comprises hydrogen or alkyl comprising from 1 to 4 carbons; and wherein p is an integer from 0-2.

In one aspect, X is Br or Cl. In a specific aspect, X is Br.

In one aspect, each of R^{13a}, R^{13b}, R^{13c}, and R^{13d} independently comprises hydrogen. In a further aspect, either R^{13a} or R^{13d} comprises methyl. In a still further aspect, neither R^{13c} nor R^{13d} comprises F.

In a further aspect, each of R^{14a} and R^{14b} independently comprises methyl.

In one aspect, R¹⁵ comprises methyl.

In a further aspect, the compound comprises a structure represented by a formula selected from:

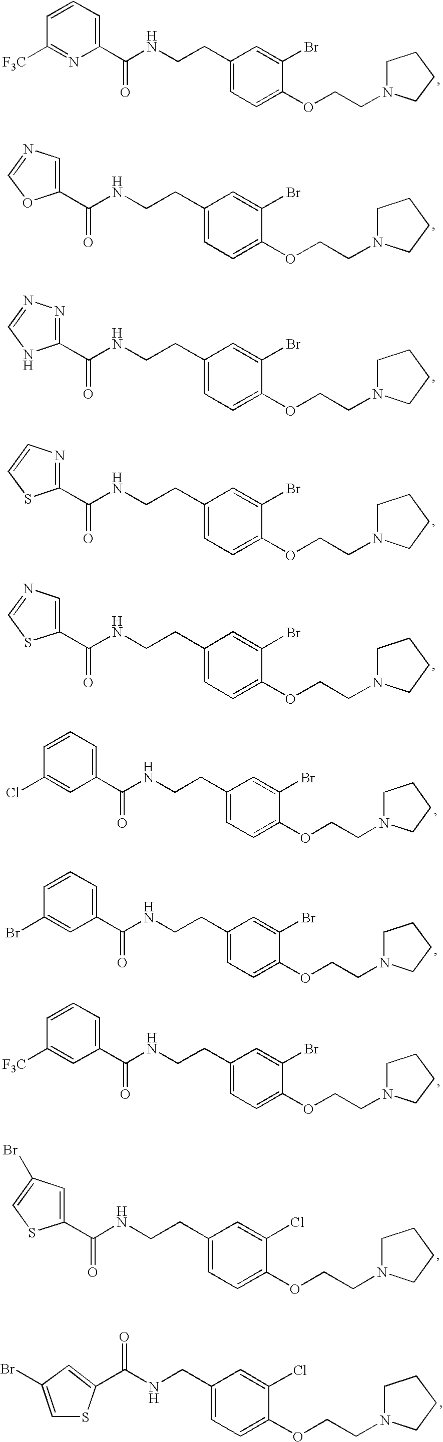

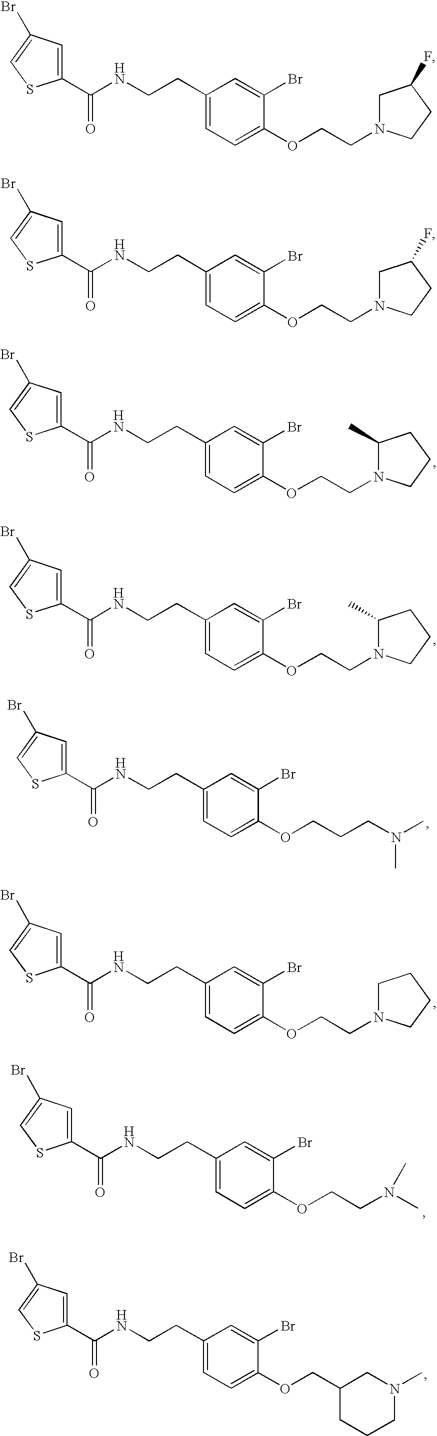

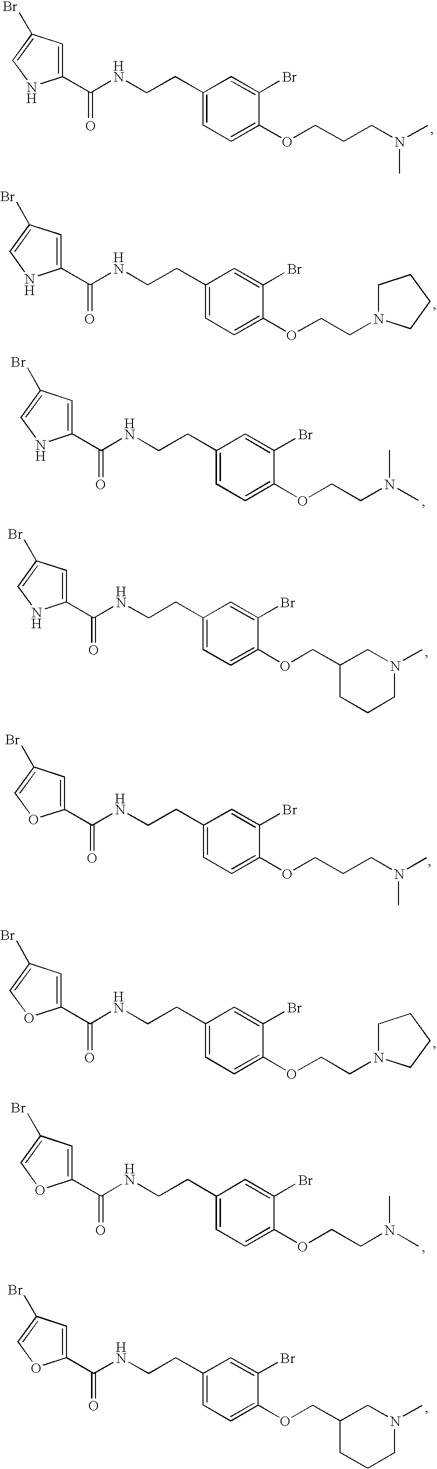

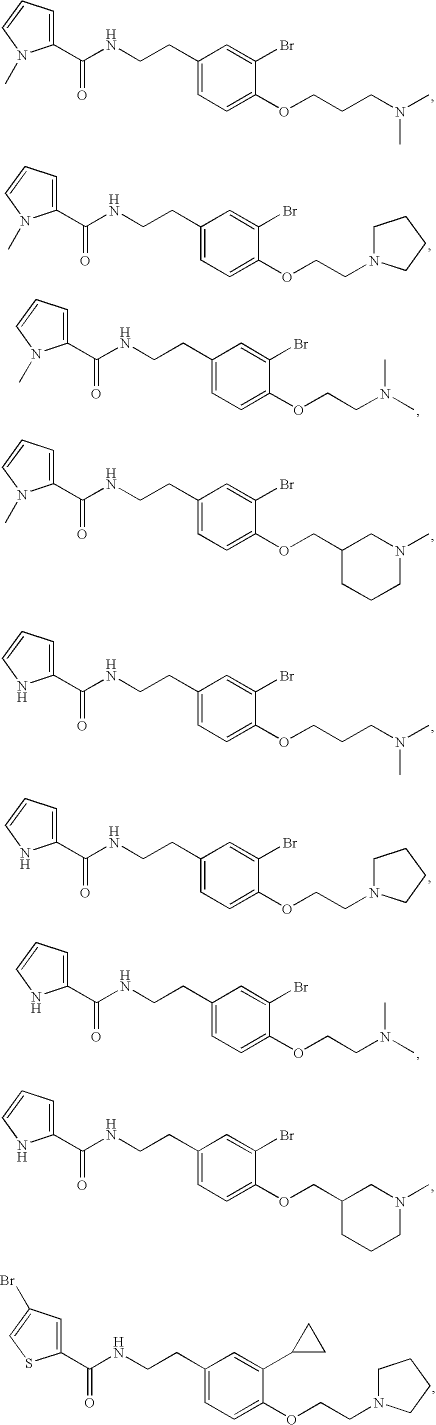

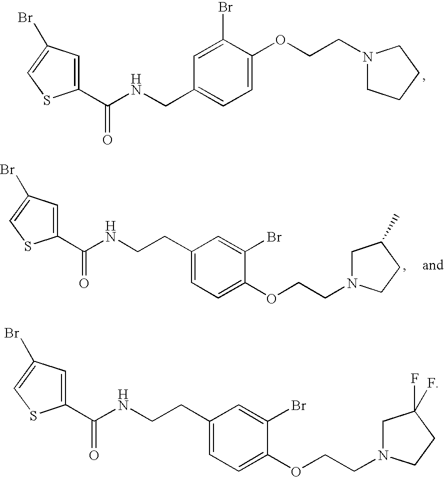

In one aspect, the compound is selected from: N-(3-bromo-4-(2-(pyrrolidin-1-yl)ethoxy)phenethyl)-6-(trifluoromethyl)picolinamide; N-(3-bromo-4-(2-(pyrrolidin-1-yl)ethoxy)phenethyl)oxazole-5-carboxamide; N-(3-bromo-4-(2-(pyrrolidin-1-yl)ethoxy)phenethyl)-4H-1,2,4-triazole-3-carboxamide; N-(3-bromo-4-(2-(pyrrolidin-1-yl)ethoxy)phenethyl)thiazole-2-carboxamide; N-(3-bromo-4-(2-(pyrrolidin-1-yl)ethoxy)phenethyl)thiazole-5-carboxamide; N-(3-bromo-4-(2-(pyrrolidin-1-yl)ethoxy)phenethyl)-3-chlorobenzamide; 3-bromo-N-(3-bromo-4-(2-(pyrrolidin-1-yl)ethoxy)phenethyl)benzamide; N-(3-bromo-4-(2-(pyrrolidin-1-yl)ethoxy)phenethyl)-3-(trifluoromethyl)benzamide; 4-bromo-N-(3-chloro-4-(2-(pyrrolidin-1-yl)ethoxy)phenethyl)thiophene-2-carboxamide; 4-bromo-N-(3-chloro-4-(2-(pyrrolidin-1-yl)ethoxy)benzyl)thiophene-2-carboxamide; (S)-4-bromo-N-(3-bromo-4-(2-(3-fluoropyrrolidin-1-yl)ethoxy)phenethyl)thiophene-2-carboxamide; (R)-4-bromo-N-(3-bromo-4-(2-(3-fluoropyrrolidin-1-yl)ethoxy)phenethyl)thiophene-2-carboxamide; (S)-4-bromo-N-(3-bromo-4-(2-(2-methylpyrrolidin-1-yl)ethoxy)phenethyl)thiophene-2-carboxamide; (R)-4-bromo-N-(3-bromo-4-(2-(2-methylpyrrolidin-1-yl)ethoxy)phenethyl)thiophene-2-carboxamide; 4-bromo-N-(3-bromo-4-(3-(dimethylamino)propoxy)phenethyl)thiophene-2-carboxamide; 4-bromo-N-(3-bromo-4-(2-(pyrrolidin-1-yl)ethoxy)phenethyl)thiophene-2-carboxamide; 4-bromo-N-(3-bromo-4-(2-(dimethylamino)ethoxy)phenethyl)thiophene-2-carboxamide; 4-bromo-N-(3-bromo-4-((1-methylpiperidin-3-yl)methoxy)phenethyl)thiophene-2-carboxamide; 4-bromo-N-(3-bromo-4-(2-(pyrrolidin-1-yl)ethoxy)phenethyl)-1H-pyrrole-2-carboxamide; 4-bromo-N-(3-bromo-4-(2-(dimethylamino)ethoxy)phenethyl)-1H-pyrrole-2-carboxamide; 4-bromo-N-(3-bromo-4-((1-methylpiperidin-3-yl)methoxy)phenethyl)-1H-pyrrole-2-carboxamide; 4-bromo-N-(3-bromo-4-(3-(dimethylamino)propoxy)phenethyl)furan-2-carboxamide; 4-bromo-N-(3-bromo-4-(2-(pyrrolidin-1-yl)ethoxy)phenethyl)furan-2-carboxamide; 4-bromo-N-(3-bromo-4-(2-(dimethylamino)ethoxy)phenethyl)furan-2-carboxamide; 4-bromo-N-(3-bromo-4-((1-methylpiperidin-3-yl)methoxy)phenethyl)furan-2-carboxamide; N-(3-bromo-4-(3-(dimethylamino)propoxy)phenethyl)-1-methyl-1H-pyrrole-2-carboxamide; N-(3-bromo-4-(2-(pyrrolidin-1-yl)ethoxy)phenethyl)-1-methyl-1H-pyrrole-2-carboxamide; N-(3-bromo-4-(2-(dimethylamino)ethoxy)phenethyl)-1-methyl-1H-pyrrole-2-carboxamide; N-(3-bromo-4-((1-methylpiperidin-3-yl)methoxy)phenethyl)-1-methyl-1H-pyrrole-2-carboxamide; N-(3-bromo-4-(3-(dimethylamino)propoxy)phenethyl)-1H-pyrrole-2-carboxamide; N-(3-bromo-4-(2-(pyrrolidin-1-yl)ethoxy)phenethyl)-1H-pyrrole-2-carboxamide; N-(3-bromo-4-(2-(dimethylamino)ethoxy)phenethyl)-1H-pyrrole-2-carboxamide; and N-(3-bromo-4-((1-methylpiperidin-3-yl)methoxy)phenethyl)-1H-pyrrole-2-carboxamide.

In a specific aspect, the compound is N-(3-bromo-4-(2-(pyrrolidin-1-yl)ethoxy)phenethyl)oxazole-5-carboxamide.

The disclosed compounds can be chiral, and it is intended that any enantiomers, as separated, pure or partially purified enantiomers or racemic mixtures thereof are included within the scope of the disclosure.

Furthermore, when a double bond or a fully or partially saturated ring system or more than one center of asymmetry or a bond with restricted rotatability is present in the molecule, diastereomers can be formed. It is intended that any diastereomers, as separated, pure or partially purified diastereomers or mixtures thereof are included within the scope of the disclosure.

Some of the disclosed compounds can exist in different tautomeric forms, and it is intended that any tautomeric forms, which the compounds are able to form, are included within the scope of the present invention.

In one aspect, disclosed are also isotopically labeled compounds, which are identical to those recited elsewhere herein, but for the fact that one or more atoms are replaced by an atom having an atomic mass or mass number different from the atomic mass or mass number usually found in nature. Examples of isotopes that can be incorporated into compounds of the present invention include, but are not limited to, isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ¹⁵O, ³¹P, ³²P, ³⁵S, ¹⁸F, and ³⁶Cl, ¹²³I, respectively.

The disclosed compounds and pharmaceutically acceptable derivatives thereof that contain the aforementioned isotopes and/or other isotopes of other atoms are within the scope of this disclosure. Certain isotopically labeled compounds, for example those into which radioactive isotopes such as ³H and ¹⁴C are incorporated, can be useful in drug and/or substrate tissue distribution assays. Tritiated, i.e., ³H, and carbon-14, i.e., ¹⁴C, isotopes are useful due to their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, can be employed in some circumstances.

2. Pharmaceutically Acceptable Derivatives

Disclosed are pharmaceutically acceptable derivatives of the compounds, as defined hereinabove. Pharmaceutically acceptable derivatives include those that increase, or allow, the bioavailability of the compounds disclosed herein when such compounds are administered to a subject (e.g., by allowing an orally administered compound to be more readily absorbed into the blood) or which enhance delivery of the parent compound to a biological compartment (e.g., the lymphatic system) relative to the parent species.

Pharmaceutically acceptable derivatives thus include among others pro-drugs. A pro-drug can be a derivative of a compound which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the compounds disclosed herein. Conventional procedures for the selection and preparation of suitable prodrug derivatives are described, for example, in “Design of Prodrugs”, ed. H. Bundgaard, Elsevier, 1985.

A pharmaceutically acceptable derivative also includes pharmaceutically acceptable salts of the disclosed compounds including those derived from pharmaceutically acceptable inorganic and organic acids and bases. Such salts include pharmaceutically acceptable metal salts, ammonium and alkylated ammonium salts, among others. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates, which the present compounds are able to form.

The acid addition salts can be obtained as the direct products of synthetic methods disclosed herein. In the alternative, the free base may be dissolved in a suitable solvent containing the appropriate acid, and the salt isolated by evaporating the solvent or otherwise separating the salt and solvent.

The disclosed compounds can form solvates with low molecular weight solvents using methods well known in the art. Such solvates are also contemplated as being within the scope of the present disclosure.

The present disclosure also encompasses active metabolites of the compounds.

3. H3 Modulators

The disclosed compounds can be modulators of H3 activity. In one aspect, the present compounds can be inhibitors, inverse agonists, or antagonists of a histamine H3 receptor. Histamine is known to play an important role in a subject, with actions spanning diverse physiological roles, from acting as a neurotransmitter in the central nervous system (CNS) to peripheral effects on gastric acid secretion and smooth muscle contraction in a mammal. Histamine H3 antagonists can lead to increased histamine levels and can consequently be useful for the treatment of a variety of disorders, including CNS disorders.

The H3 modulating ability of a disclosed compound can be determined by routine methods known in the art. For example, affinity (binding) and inhibition studies can be employed to determine H3 modulating ability.

In one aspect, H3 affinity (Kᵢ) and inhibition (IC₅₀) can be determined in 5-point concentration response curves. These values can be determined for a variety of compounds, such as those listed in Table 1, for example. Using data obtained from such methods, SAR can be established. In general, the nature of the heterocyclic carboxylic acid incorporated into analogues, such as those shown in Table 1, can have little effect on H3 affinity/inhibition. By contrast, structural variations in chloroalkyl amines employed can have impact. For instance, the morpholino ethyl congeners can lose 12- to 30-fold in terms of both H3 affinity and inhibition relative to 4 (Kᵢs and IC₅₀s in the 12 μM to 73 uM range).

In a further aspect, analogues incorporating a truncated N,N-dimethylamino ethyl chain can vary little in activity from those containing the natural N,N-dimethylamino propyl chain. Similarly, analogues containing a cyclic constraint in the aminoalkly tether can be approximately equivalent to 4 in activity. Analogues with an ethyl pyrrolidine moiety can display improved H3 affinity and inhibition relative to natural dispyrin (4), and can provide submicromolar activities. It should be appreciated that such an exercise in molecular editing can improve both H3 affinity and inhibition about 13-fold relative to dispyrin (4), and provide another example of an unnatural product with biological activity beyond the natural product.

TABLE 1

| STRUCTURE | H3 Kᵢ (μM) | H3 IC₅₀ (μM) |
|---|---|---|
| 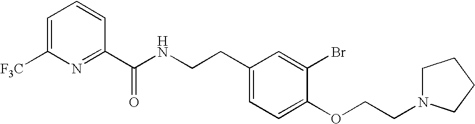 | 0.27 | 0.56 |
| 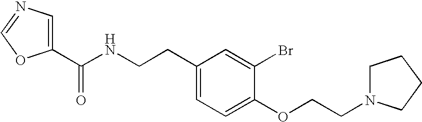 | 0.037 | 0.083 |
| 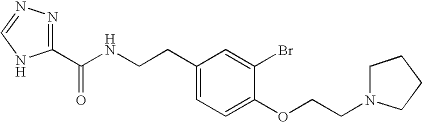 | 0.43 | 0.97 |
| 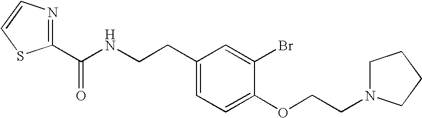 | 0.116 | 0.26 |
| 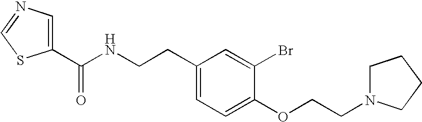 | 0.032 | 0.072 |
| 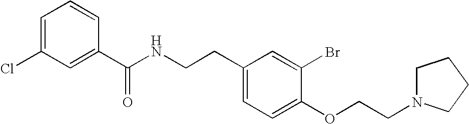 | 0.25 | 0.55 |
| 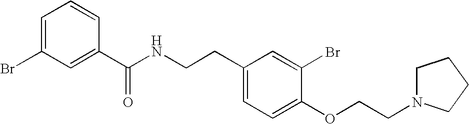 | 0.21 | 0.45 |
| 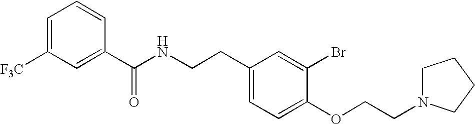 | 0.44 | 0.98 |
| 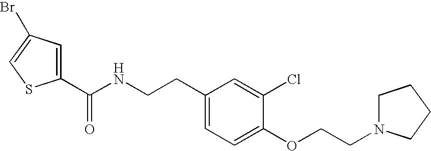 | 0.16 | 0.35 |
| 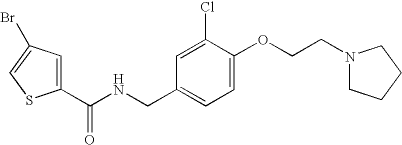 | 1.1 | 2.4 |
| 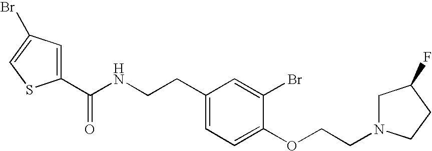 | 1.1 | 2.5 |
| 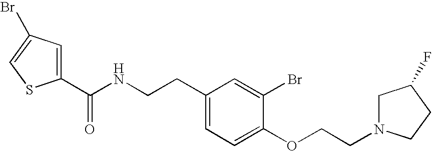 | 1.1 | 2.1 |
| 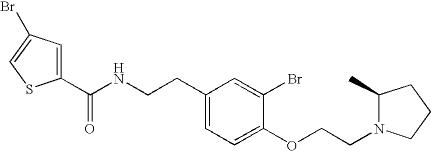 | 0.33 | 0.75 |
| 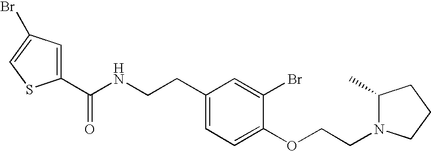 | 0.31 | 0.71 |
| 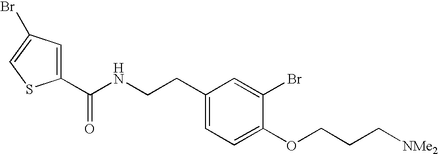 | 1.6 | 3.6 |
| 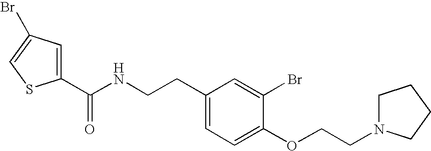 | 0.08 | 0.18 |
| 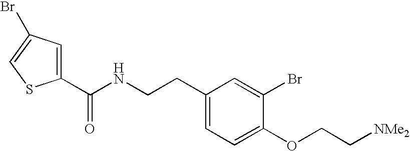 | 0.67 | 1.5 |
| 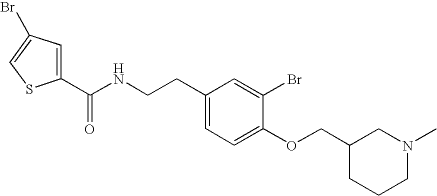 | 1.2 | 1.7 |
| 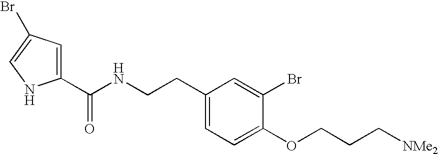 | 1.1 | 2.4 |
| 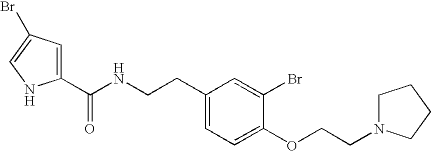 | 0.19 | 0.43 |
| 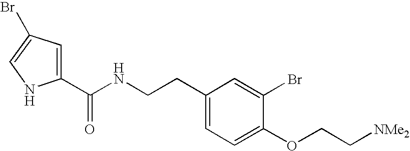 | 1.6 | 3.7 |
| 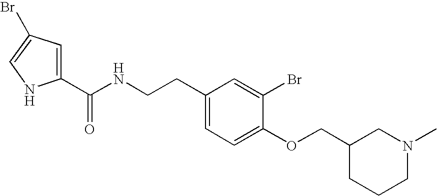 | 1.3 | 2.9 |
| 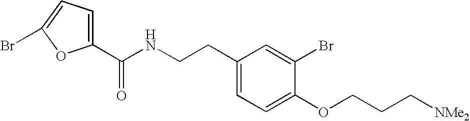 | 2.2 | 4.8 |
| 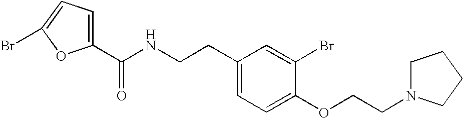 | 0.17 | 0.39 |
| 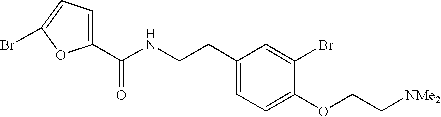 | 2.7 | 5.9 |
| 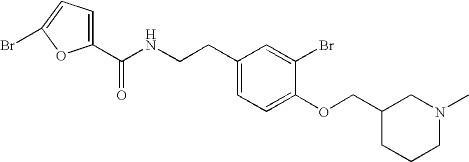 | 2.1 | 4.5 |
| 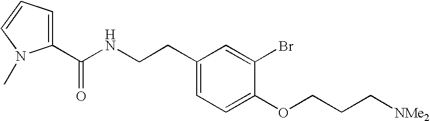 | 1.7 | 3.9 |
| 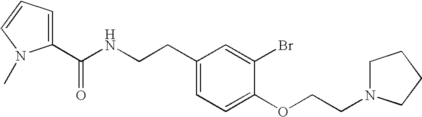 | 0.13 | 0.39 |
| 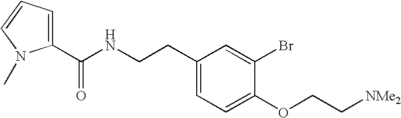 | 2.1 | 4.5 |
| 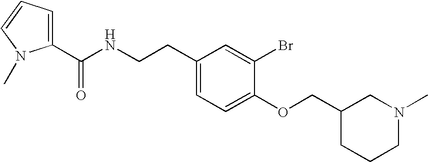 | 0.91 | 2.1 |
| 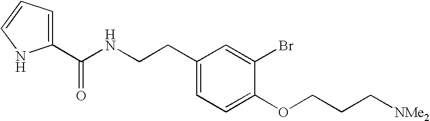 | 1.3 | 2.9 |
| 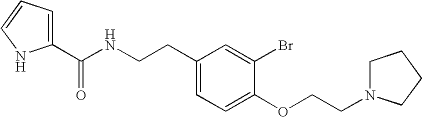 | 0.15 | 0.38 |
| 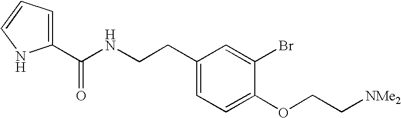 | 1.5 | 3.4 |
| 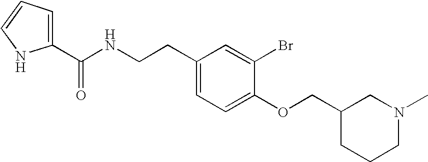 | 1.5 | 3.6 |

It is understood that the compounds can be used in connection with the methods and compositions disclosed herein.

### C. METHODS OF MAKING THE COMPOUNDS

In one aspect, the invention relates to methods of making compounds useful as H3 inhibitors, antagonists, or inverse agonists, as disclosed herein, which can be useful in the treatment of disorders associated with histamine H3 activity.

The disclosed compounds can be prepared by several processes well known in the art. For example, a variety of aryl and heteroaryl carboxylic acids can be coupled, using peptide coupling chemistry, to various aryl or heteroaryl amines, followed by an optional Williamson ether synthesis to further functionalize the coupled amide.

In one aspect, disclosed are methods of preparing a compound comprising the step of reacting a compound comprising a structure represented by a formula:

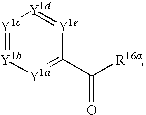

wherein each of Y^{1a}, Y^{1b}, Y^{1c}, Y^{1d}, and Y^{1e} is independently selected from nitrogen or CR¹¹, wherein each R¹¹, when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons; wherein R^{16a} comprises OH, alkoxy, acyloxy, hydrogen, or halogen; with the proviso that no more than two of Y^{1a}, Y^{1b}, Y^{1c}, Y^{1d}, and Y^{1e} are nitrogen; or

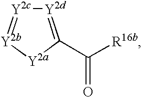

wherein Y^{2a} is selected from O, S, and NR¹², wherein R¹², if present, is selected from hydrogen or an alkyl residue comprising from 1 to 4 carbons; wherein each of Y^{2b}, Y^{2c}, and Y^{2d} is independently selected from N and CR¹², wherein each R¹², when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons; wherein R^{16b} comprises OH, alkoxy, acyloxy, hydrogen, or halogen; with the proviso that no more than three of Y^{2a}, Y^{2b}, Y^{2c}, and Y^{2d} are heteroatom; with a compound having a structure represented by a formula:

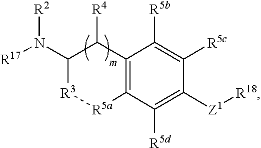

wherein R² is selected from hydrogen, an optionally substituted organic residue comprising from 1 to 6 carbons, or a hydrolysable residue; wherein each of R³, and R⁴ (if present) independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein ---- is an optional covalent bond; wherein m an integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; wherein R¹⁷ is hydrogen or a hydrolyzable group; wherein R¹⁸ is hydrogen, a hydrolyzable group, a protecting group, or an optionally substituted organic residue comprising from 1 to 12 carbons; thereby forming an amide bond.

In one aspect, wherein R^{16a} or R^{16b} is OH, reacting further comprises the step of activating a carboxylic acid, thereby forming an electrophile prior to forming the amide bond. In this aspect, the step of activating the carboxylic acid comprises reacting the carboxylic acid with a peptide coupling reagent. In a specific aspect, the peptide coupling reagent comprises one or more of BOP, DIC, HOBt, CDI, DCC, EDC, HATU, HBTU, HOOBt, HCTU, PyBOP, TATU, and TBTU.

In one aspect, R¹⁸ comprises a structure represented by a formula:

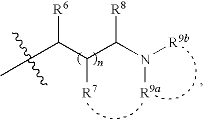

wherein n is an integer selected from 0, 1, and 2; wherein R⁶, R⁷ (if present), and R⁸ independently comprises two residues independently selected from hydrogen and an optionally substituted organic residue comprising from 1 to 6 carbons; and wherein each of R^{9a} and R^{9b} independently comprises hydrogen or an optionally substituted organic residue comprising from 1 to 12 carbons.

In a further aspect, a synthetic method useful for preparing the disclosed compounds can be represented by the following synthetic scheme:

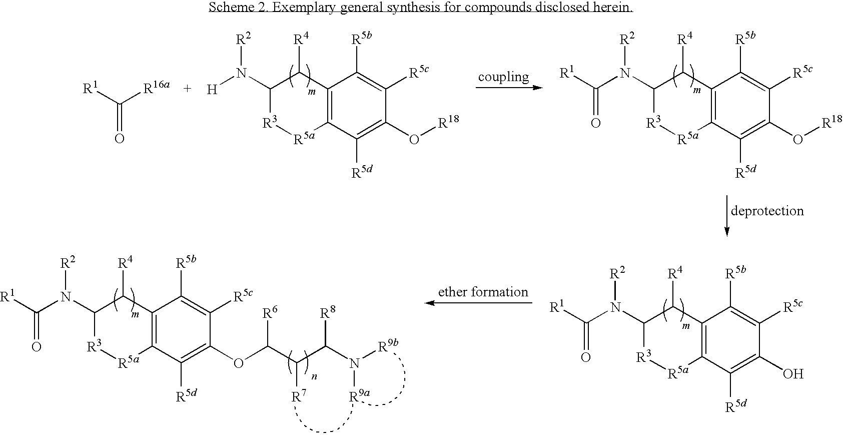

As discussed herein above, the first step shown in exemplary Scheme 1 is a coupling step. Such a coupling step can be accomplished by methods known in art, including but not limited to peptide coupling methods. In general, a peptide coupling method can be carried out by first activating a carboxylic acid with a coupling reagent (e.g., DIC, DCC, etc.) followed by amide bond formation. Alternatively, an acid halide or other suitable electrophile could be used in lieu of a carboxylic acid.

In one aspect, a deprotection step, if R¹⁸ is alkyl, can comprise the use of a Lewis acid, such as, for example, BX₃, wherein X is halide. An exemplary Lewis acid is a BBr₃. Depending on the strength of such a Lewis acid, a deprotection step can be carried out at reduced temperatures, e.g., about −78° C.

In a further aspect, the step of ether formation can comprise the use of methods known in the art, generally referred to as a Williamson synthesis, wherein a nucleophile (e.g., a deprotonated phenol) reacts with an aliphatic electrophile (e.g., an alkyl halide).

In one aspect, the compounds disclosed herein, when existing as a diastereomeric or enantiomeric mixture, can be separated into diastereomeric pairs of enantiomers by, for example, fractional crystallization from a suitable solvent, for example methanol or ethyl acetate or a mixture thereof. Alternatively, known chiral HPLC methods can be used to separate diastereomers or enantiomers. A pair of enantiomers thus obtained can be separated into individual isomers by conventional means, for example by the use of an optically active acid as a resolving agent. Alternatively, any enantiomer of a compound of the formula can be obtained by stereospecific synthesis using optically pure starting materials or reagents of known configuration or through enantioselective synthesis.

In a further aspect, certain materials, compounds, compositions, and components disclosed herein can be obtained commercially or readily synthesized using techniques generally known to those of skill in the art. For example, the starting materials and reagents used in preparing the disclosed compounds and compositions can be available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

In a still further aspect, the disclosed methods provide a disclosed compound, for example, a compound listed in Table 2. Compounds in Table 2 were synthesized with methods identical or analogous to those shown herein. The requisite starting materials were commercially available, described in the literature, or readily synthesized by one skilled in the art of organic synthesis.

TABLE 2

| | | MS + |
|---|---|---|
| STRUCTURE | NOMENCLATURE | 1 |
| 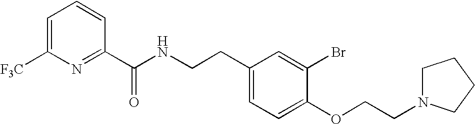 | N-(3-bromo-4-(2-(pyrrolidin-1- yl)ethoxy)phenethyl)-6- (trifluoromethyl)picolinamide | 486.1 |
| 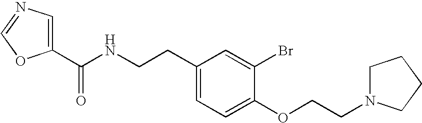 | N-(3-bromo-4-(2-(pyrrolidin-1- yl)ethoxy)phenethyl)oxazole-5-carboxamide | 408.1 |
| 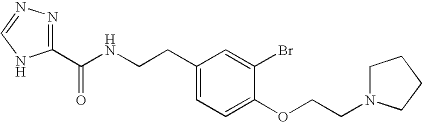 | N-(3-bromo-4-(2-(pyrrolidin-1-yl)ethoxy)phenethyl)- 4H-1,2,4-triazole-3-carboxamide | 408.1 |
| 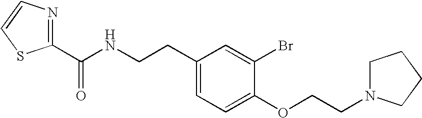 | N-(3-bromo-4-(2-(pyrrolidin-1-yl)ethoxy) phenethyl)thiazole-2-carboxamide | 423.1 |
| 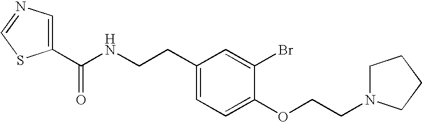 | N-(3-bromo-4-(2-(pyrrolidin-1- yl)ethoxy)phenethyl)thiazole-5-carboxamide | 423.1 |
| 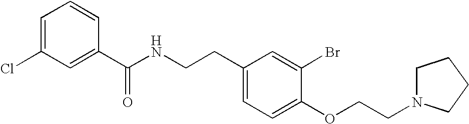 | N-(3-bromo-4-(2-(pyrrolidin-1-yl)ethoxy) phenethyl)-3-chlorobenzamide | 451.1 |
| 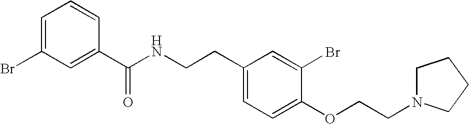 | 3-bromo-N-(3-bromo-4-(2-(pyrrolidin-1- yl)ethoxy)phenethyl)benzamide | 495.1 |
| 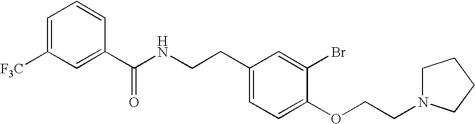 | N-(3-bromo-4-(2-(pyrrolidin-1-yl)ethoxy)phenethyl)-3- (trifluoromethyl)benzamide | 485.1 |
| 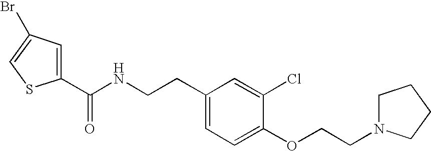 | 4-bromo-N-(3-chloro-4-(2-(pyrrolidin-1-yl)ethoxy) phenethyl)thiophene-2-carboxamide | 457.1 |
| 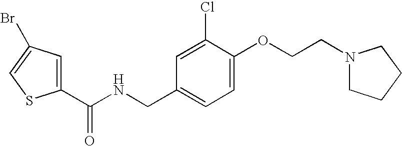 | 4-bromo-N-(3-chloro-4-(2-(pyrrolidin-1-yl)ethoxy) benzyl)thiophene-2-carboxamide | 443.1 |
| 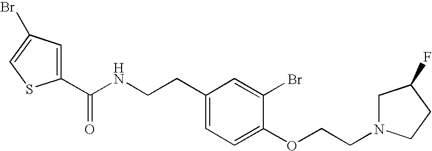 | (S)-4-bromo-N-(3-bromo-4-(2-(3-fiuoropyrrolidin-1- yl)ethoxy)phenethyl)thiophene-2-carboxamide | 518.9 |
| 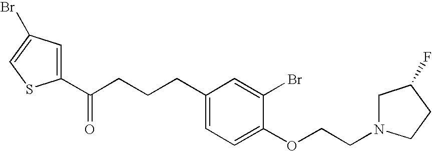 | (R)-4-(3-bromo-4-(2-(3-fluoropyrrolidin-1-yl)ethoxy)phenyl)-1- (4-bromothiophen-2-yl)butan-1-one | 518.9 |
| 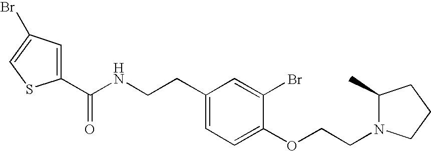 | (S)-4-bromo-N-(3-bromo-4-(2-(2-methylpyrrolidin-1- yl)ethoxy)phenethyl)thiophene-2-carboxamide | 514.9 |
| 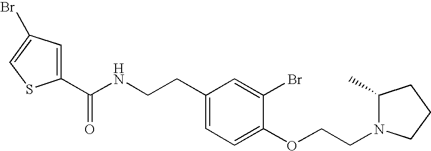 | (R)-4-bromo-N-(3-bromo-4-(2-(2-methylpyrrolidin-1- yl)ethoxy)phenethyl)thiophene-2-carboxamide | 514.9 |
| 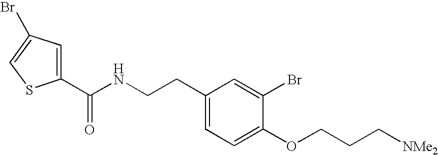 | 4-bromo-N-(3-bromo-4-(3-(dimethylamino) propoxy)phenethyl)thiophene-2-carboxamide | 488.9 |
| 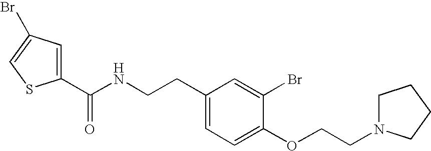 | 4-bromo-N-(3-bromo-4-(2-(pyrrolidin-1- yl)ethoxy)phenethyl)thiophene-2-carboxamide | 500.9 |
| 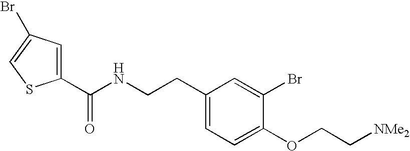 | 4-bromo-N-(3-bromo-4-(2- (dimethylamino)ethoxy)phenethyl)thiophene-2- carboxamide | 474.9 |
| 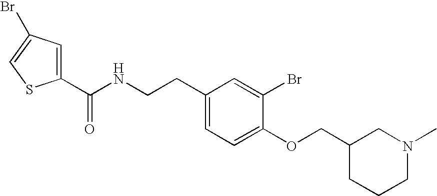 | 4-bromo-N-(3-bromo-4-((1-methylpiperidin-3- yl)methoxy)phenethyl)thiophene-2-carboxamide | 514.9 |
| 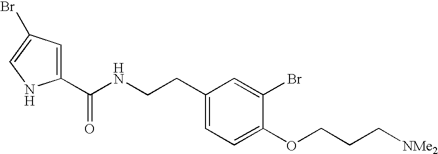 | 4-bromo-N-(3-bromo-4-(3-(dimethylamino) propoxy)phenethyl)-1H-pyrrole-2-carboxamide | 472.0 |
| 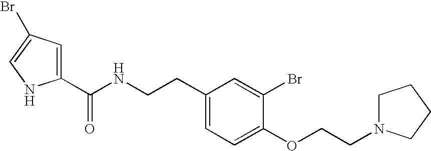 | 4-bromo-N-(3-bromo-4-(2-(pyrrolidin-1-yl) ethoxy)phenethyl)-1H-pyrrole-2-carboxamide | 483.0 |
| 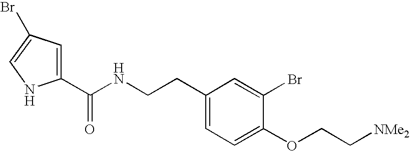 | 4-bromo-N-(3-bromo-4-(2-(dimethylamino) ethoxy)phenethyl)-1H-pyrrole-2-carboxamide | 458.0 |
| 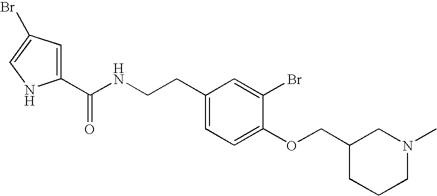 | 4-bromo-N-(3-bromo-4-((1-methylpiperidin-3-yl) methoxy)phenethyl)-1H-pyrrole-2-carboxamide | 498.0 |
| 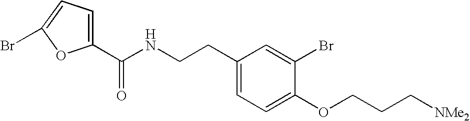 | 5-bromo-N-(3-bromo-4-(3-(dimethylamino) propoxy)phenethyl)furan-2-carboxamide | 473.1 |
| 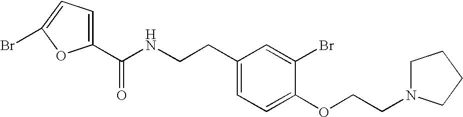 | 5-bromo-N-(3-bromo-4-(2-(pyrrolidin-1- yl)ethoxy)phenethyl)furan-2-carboxamide | 485.0 |
| 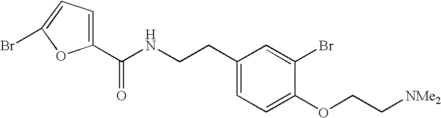 | 5-bromo-N-(3-bromo-4-(2-(dimethylamino) ethoxy)phenethyl)furan-2-carboxamide | 458.9 |
| 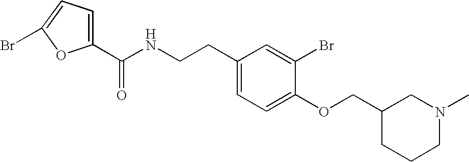 | 5-bromo-N-(3-bromo-4-((1-methylpiperidin-3- yl)methoxy)phenethyl)furan-2-carboxamide | 499.0 |
| 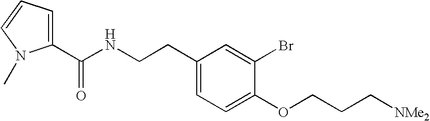 | N-(3-bromo-4-(3-(dimethylamino)propoxy) phenethyl)-1-methyl-1H-pyrrole-2-carboxamide | 408.1 |
| 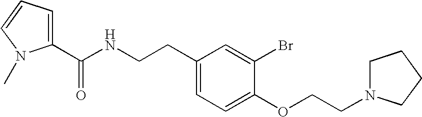 | N-(3-bromo-4-(2-(pyrrolidin-1-yl)ethoxy)phenethyl)- 1-methyl-1H-pyrrole-2-carboxamide | 420.1 |
| 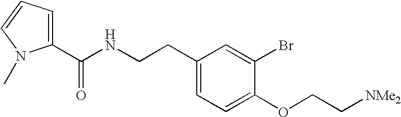 | N-(3-bromo-4-(2-(dimethylamino)ethoxy)phenethyl)- 1-methyl-1H-pyrrole-2-carboxamide | 394.1 |
| 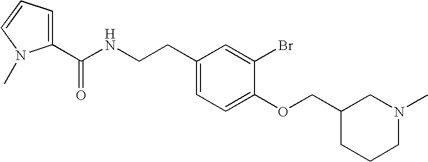 | N-(3-bromo-4-((1-methylpiperidin-3-yl)methoxy) phenethyl)-1-methyl-1H-pyrrole-2-carboxamide | 434.1 |
| 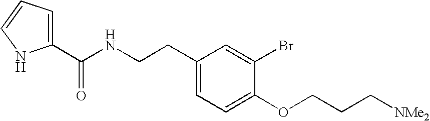 | N-(3-bromo-4-(3-(dimethylamino)propoxy) phenethyl)-1H-pyrrole-2-carboxamide | 394.1 |
| 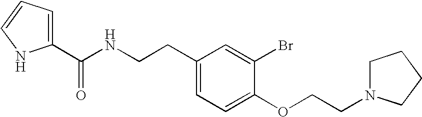 | N-(3-bromo-4-(2-(pyrrolidin-1-yl)ethoxy) phenethyl)-1H-pyrrole-2-carboxamide | 406.1 |
| 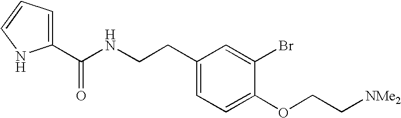 | N-(3-bromo-4-(2-(dimethylamino)ethoxy) phenethyl)-1H-pyrrole-2-carboxamide | 380.1 |
| 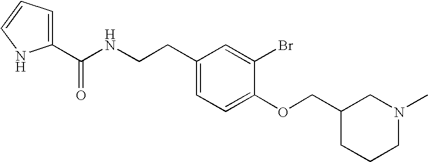 | N-(3-bromo-4-((1-methylpiperidin-3-yl)methoxy) phenethyl)-1H-pyrrole-2-carboxamide | 420.1 |

Also disclosed are the products of any of the disclosed synthetic methods.

### D. METHODS OF USING THE COMPOSITIONS

In one aspect, disclosed are methods of using compounds comprising a structure represented by a formula:

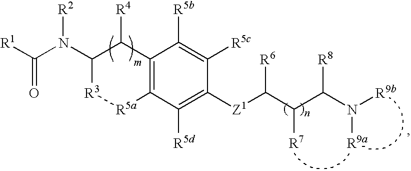

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof, with the proviso that the compound is not Dispyrin, Purealidin Q, Purealidin S, Purpurealidin A, Purpurealidin B, or Fistularin-3; with a pharmaceutically acceptable carrier.

In a further aspect, disclosed are methods of using one or more disclosed compounds.

The disclosed compounds can interact with a G-protein coupled receptor, including the histamine H3 receptor, and are accordingly useful for the treatment of a wide variety of conditions and disorders in which G-protein coupled receptor interactions are beneficial.

In one aspect, disclosed is a method of modulating the activity of a G-protein coupled receptor in at least one cell comprising the step of contacting the at least one cell with at least one compound as disclosed herein, thereby modulating activity of the G-protein coupled receptor in the at least one cell.

In a further aspect, the step of contacting at least one cell can occur in vivo, ex vivo, or in vitro using methods known in the art. The location and/or affinity of a compound disclosed herein, once on or inside a cell, can be determined using methods known in the art, such as, for example, radioligand binding assays, fluorescence assays, and the like.

1. Subjects

In a further aspect, disclosed is a method of modulating the activity of a G-protein coupled receptor in a subject in need thereof comprising the step of administering to the subject a therapeutically effective amount of at least one compound as disclosed herein, or a pharmaceutically acceptable derivative or N-oxide thereof, thereby modulating activity of the G-protein coupled receptor in the subject.

In one aspect, the G-protein coupled receptor is an H3 receptor. In further aspect, the G-protein coupled receptor is a mammalian H3 receptor, including a human H3 receptor. Thus, in various aspects, a suitable subject can include an animal, such as a mammal, including a human.

In a further aspect, disclosed is a method for treating a disorder associated with G-protein coupled receptor activity in a subject comprising the step of administering to the subject a therapeutically effective amount of at least one compound as disclosed herein, or a pharmaceutically acceptable derivative or N-oxide thereof, thereby treating the disorder in the subject. In a specific aspect, the disorder can be associated with H3 activity.

In one aspect, the subject can be diagnosed with the disorder prior to the administering step. In a further aspect, the method of administering can further comprise the step of identifying a subject with the disorder. Suitable subjects include those that have been diagnosed with a need for inhibition of H3 receptor activity prior to the administering step, for example.

In general, a subject can be any age, including a fetus. A subject to which a compound or compositions disclosed herein can be administered can be an animal, including but not limited to a mammal, such as a non-primate (e.g., cows, pigs, sheep, goats, horses, chickens, dogs, rats, etc.) and a primate (e.g., a monkey such as a acynomolgous monkey and a human). A subject can also be a laboratory animal (e.g, a mouse, rabbit, guinea pig, fruit fly, etc.).

2. Treating a Disorder

In various aspects, the disorder is a neurological disorder associated with G-protein couple receptor activity dysfunction, including histamine H3 receptor dysfunction. Examples of such disorders include alcohol addiction or dependency, atherosclerosis, hypertension, IGT (impaired glucose tolerance), diabetes, dyslipidaemia, coronary heart disease, gallbladder disease, osteoarthritis, cancer including endometrial cancer, breast, prostate and colon cancers, obesity, bulimia, binge eating, conditions associated with epilepsy, motion sickness, vertigo, dementia, Alzheimer's disease, allergic rhinitis, ulcer, anorexia, migraine hyperactivity disorder, schizophrenia, obesity, ADHD, cognitive disorders, depression, anxiety, physchoses, Tourette's syndrome, sexual dysfunction, drug addiction, drug abuse, senile dementia, obsessive-compulsive behavior, panic attacks, pain, social phobias, non-insulin dependent diabetes mellitus, hyperglycemia, tardive dyskinesia, Parkinson's disease, constipation, arrhythmia, disorders of the neuroendrocrine system, stress, and spasticity, as well as acid secretin, ulcers, airway constriction, asthma, allergy, inflammation, and prostate dysfunction.

Anxiety disorders include, for example, generalized anxiety disorder, panic disorder, PTSD, and social anxiety disorder. Mood disorders include, for example, depressed mood, mixed anxiety and depressed mood, disturbance of conduct, and mixed disturbance of conduct and depressed mood. Cognitive disorders include, for example, in addition to ADHD, attention-deficit disorder (ADD) or other attention adjustment or Cognitive disorders due to general medical conditions. Psychotic disorders include, for example, schizoaffective disorders and schizophrenia, sleep disorders include, for example, narcolepsy and enuresis.

Depression can include, for example, depression in cancer patients, depression in Parkinson's patients, post-myocardial infarction depression, depression in patients with human immunodeficiency virus (HIV), Subsyndromal Symptomatic depression, depression in infertile women, pediatric depression. major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including, for example, agoraphobia, social phobia or simple phobias. Eating disorders can include, for example, anorexia nervosa or bulimia nervosa. Chemical dependencies can include, for example, addictions to alcohol, cocaine, amphetamine and other psychostimulants, morphine, heroin and other opioid agonists, phenobarbital and other barbiturates, nicotine, diazepam, benzodiazepines and other psychoactive substances. Parkinson's diseases can include, for example, dementia in Parkinson's disease, neuroleptic-induced parkinsonism or tardive dyskinesias. Headache can include, for example, headache associated with vascular disorders; withdrawal syndrome. Age-associated learning and mental disorders can include, for example, apathy, bipolar disorder, chronic fatigue syndrome, chronic or acute stress, conduct disorder, cyclothymic disorder. Somatoform disorders can also be treated, such as somatization disorder, conversion disorder, pain disorder, hypochondriasis, body dysmorphic disorder, undifferentiated disorder, and somatoform NOS, and incontinence.

Inhalation disorders can also be treated, as well as intoxication disorders, mania, oppositional defiant disorder, peripheral neuropathy; post traumatic stress disorder, late luteal phase dysphoric disorder, specific developmental disorders, SSRI “poop out” syndrome, or a patient's failure to maintain a satisfactory response to SSRI therapy after an initial period of satisfactory response, and tic disorders including Tourette's disease.

3. Co-Therapeutic Use

Compounds disclosed herein can also be used in combination with other therapeutic agents, for example histamine H1 antagonists or medicaments claimed to be useful as either disease modifying or symptomatic treatments of Alzheimer's disease. Suitable examples of other therapeutic agents can be agents known to modify cholinergic transmission such as 5-HT₆ antagonists, M1 muscarinic agonists, M2 muscarinic antagonists or acetylcholinesterase inhibitors. When the compounds are used in combination with other therapeutic agents, the compounds can be administered either sequentially or simultaneously by any convenient route, as further discussed herein.

The disclosed compounds can also be used as part of a combination therapy, including their administration as separate entities or combined in a single delivery system, which employs an effective dose of a histamine H3 antagonist compound disclosed herein and an effective dose of a histamine H1 antagonist, such as cetirizine (ZYRTEC™), for the treatment of allergic rhinitis, nasal congestion and allergic congestion.

The disclosed compounds can also be used as part of a combination therapy, which employs an effective dose of a histamine H3 antagonist compound disclosed herein and an effective dose of a neurotransmitter reuptake blocker. Examples of neurotransmitter reuptake blockers can include the serotonin-selective reuptake inhibitors (SSRI's) like sertraline (ZOLOFT™), fluoxetine (PROZAC™), and paroxetine (PAXIL™), or non-selective serotonin, dopamine or norepinephrine reuptake inhibitors for treating depression and mood disorders.

The disclosure thus provides, in various aspects, a combination comprising a compound disclosed herein or a pharmaceutically acceptable derivative thereof together with a further therapeutic agent or agents. For example, a combination can be provided as a kit comprising a compound disclosed herein or a pharmaceutically acceptable derivative thereof together with a further therapeutic agent or agents. The kits can be co-packaged, co-formulated, and/or co-delivered with the further therapeutic agent or agents. For example, a drug manufacturer, a drug reseller, a physician, or a pharmacist can provide a disclosed kit for delivery to a patient.

4. Methods of Preparing a Medicament

Disclosed are also methods of preparing a medicament comprising the step of combining one or more compounds comprising a structure represented by a formula:

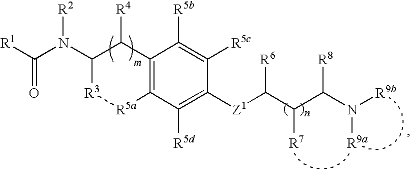

wherein R¹ is selected from optionally substituted aryl, heteroaryl, cycloalkyl, heterocycloalkyl, cycloalkenyl, or heterocycloalkenyl; wherein R² is selected from hydrogen, an optionally substituted organic residue comprising from 1 to 6 carbons, or a hydrolysable residue; wherein each of R³, R⁴ (if present), R⁶, R⁷ (if present), and R⁸ independently comprises two residues independently selected from hydrogen and an optionally substituted organic residue comprising from 1 to 6 carbons; wherein Z¹ is O, S, or NR¹⁰, wherein R¹⁰, when present, is hydrogen or an optionally substituted organic residue comprising from 1 to 12 carbons; wherein each ---- is, independently, an optional covalent bond; wherein m and n are, independently, integers selected from 0, 1, and 2; wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and wherein each of R^{9a} and R^{9b} independently comprises hydrogen or an optionally substituted organic residue comprising from 1 to 12 carbons; or a pharmaceutically acceptable derivative or N-oxide thereof, with the proviso that the compound is not Dispyrin, Purealidin Q, Purealidin S, Purpurealidin A, Purpurealidin B, or Fistularin-3; with a pharmaceutically acceptable carrier.

In one aspect, disclosed are methods of preparing a medicament comprising the step of combining one or more disclosed compounds with a pharmaceutically acceptable carrier.

### E. PHARMACEUTICAL COMPOSITIONS AND ADMINISTRATION OF SAME

The compounds disclosed herein can be administered alone or in combination with pharmaceutically acceptable carriers or excipients, in either single or multiple doses. The pharmaceutical compositions can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In one aspect, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered.

Suitable pharmaceutical carriers include inert solid diluents or fillers, sterile aqueous solution and various organic solvents. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Similarly, the carrier or diluent may include any sustained release material known in the art, such as glyceryl monostearate or glyceryl distearate, alone or mixed with a wax. The pharmaceutical compositions formed by combining the compounds disclosed herein and the pharmaceutically acceptable carriers can then be readily administered in a variety of dosage forms suitable for the disclosed routes of administration. The formulations can conveniently be presented in unit dosage form by methods known in the art of pharmacy.

Examples of various suitable pharmaceutical carriers are described in “Remington's Pharmaceutical Sciences” by E. W. Martin. Such compositions can contain a therapeutically effective amount of one or more active compounds disclosed herein together with a suitable amount of carrier so as to provide the form for proper administration to the subject. The formulation should typically suit the mode of administration.

Pharmaceutical compositions can be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

For parenteral administration, solutions of the compounds in sterile aqueous solution, aqueous propylene glycol or sesame or peanut oil can be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Toxicity and therapeutic efficacy of the compounds and compositions disclosed herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices can be desirable. While compounds that exhibit toxic side effects can be used, care should be taken to design a delivery system that targets such compounds to the site of affected tissue in order to minimize potential damage to uninfected cells and, thereby, reduce side effects.

Data obtained from the cell culture assays and animal studies can be used in formulating a range of dosage for use in humans. The dosage of such compounds lies preferably within a range of circulating concentrations that include the ED₅₀ with little or no toxicity. Dosages can vary within this range depending upon the dosage form employed and the route of administration utilized. For any compound used in disclosed herein, the therapeutically effective dose can be estimated initially from cell culture assays. A dose can be formulated in animal models to achieve a circulating plasma concentration range that includes the IC₅₀ as determined in cell culture experiments. Such information can be used to more accurately determine useful doses in humans. Levels in plasma can be measured, for example, by high performance liquid chromatography.

Suitable daily doses for the treatment or prevention of a disorder described herein can be readily determined by those skilled in the art. A recommended dose of a compound of a compound disclosed herein can be from about 0.1 mg to about 100 mg per day, per kg of body weight, given as a single once-a-day dose in the morning or as divided doses throughout the day.

It is understood that the pharmaceutical compositions can be used in connection with the methods and compounds disclosed herein.

### F. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of disclosed herein are illustrated in the following Examples. Starting materials and the requisite intermediates are in some cases commercially available, or can be prepared according to literature procedures or as illustrated herein. All reactions were carried out under an argon atmosphere employing standard chemical techniques. Solvents for extraction, washing and chromatography were HPLC grade. All reagents were purchased from Aldrich Chemical Co. at the highest commercial quality and were used without purification. Microwave-assisted reactions were conducted using a Biotage Initiator-60. All NMR spectra were recorded on a 400 MHz Bruker AMX NMR. ¹H chemical shifts are reported in 6 values in ppm downfield from TMS as the internal standard in DMSO. Data are reported as follows: chemical shift, multiplicity (s=singlet, d=doublet, t=triplet, q=quartet, br=broad, m=multiplet), integration, coupling constant (Hz). ¹³C chemical shifts are reported in δ values in ppm with the DMSO carbon peak set to 39.5 ppm. Low resolution mass spectra were obtained on an Agilent 1200 LCMS with electrospray ionization. High resolution mass spectra were recorded on a Waters QToF-API-US plus Acquity system with electrospray ionization. Analytical thin layer chromatography was performed on 250 μM silica gel 60 F₂₅₄ plates. Merck silica gel (60, particle size 0.040-0.063 mm) was used for flash column chromatography. Analytical HPLC was performed on an Agilent 1200 analytical LCMS with UV detection at 214 nm and 254 nm along with ELSD detection. Preparative purification of library compounds was performed on a custom Agilent 1200 preparative LCMS with collection triggered by mass detection. All yields refer to analytically pure and fully characterized materials (¹H NMR, ¹³C NMR analytical LCMS and Hi-Res MS).

1. Dispyrin

The synthesis of Dispyrin can be represented by the following synthetic scheme:

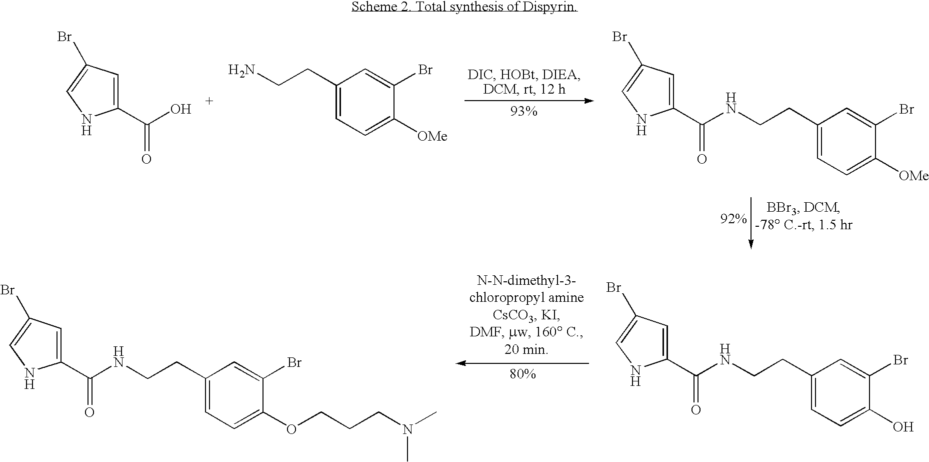

As shown in Scheme 2, the synthesis began with commercially available acid, which was coupled to 3-bromo-4-methoxyphenethylyamine to provide the product in 93% yield. Subsequent deprotection of the methyl ether with BBr₃ afforded the subsequent product (92% yield). The phenol was then alklyated with N,N-dimethyl-3-chloropropyl amine, under a microwave-assisted protocol, to deliver dispyrin 4 in 80% isolated yield. Thus, the total synthesis of dispyrin 4 was completed on a one gram scale in three synthetic steps (overall yield of 68.4%).

2. Determining Biological Activity of Dispyrin

Multiple screening avenues were pursued to identify the biological activity of dispyrin. Dispyrin was submitted to the screening deck of the Molecular Library Screening Network (MLSCN) and was evaluated against several GPCR targets (M1, M4, D1/D5, mGluR5, mGluR4) in agonist, antagonist and potentiator mode. Utilizing panels of radioligand binding assays from several companies, dispyrin was evaluated against >200 discrete molecular targets over the course of two months. The MDS Pharma Services panel identified multiple activities for dispyrin. In the initial screen at a single 10 μM concentration, dispyrin was found to provide modest inhibition (50-60% at 10 μM) of a number of calcium and potassium ion channels, including hERG. Other modest activities at the 50-60% range at 10 u.M included other GPCRs and ion channels, but none with significant activity (no Kᵢs or IC₅₀s<10 μM).

Dispyrin was found to be possess affinity for the human H3 receptor in a radioligand binding assay employing [³H](R)-α-methylhistamine, and was a moderately potent human H3 antagonist (IC₅₀=2.35 μM).

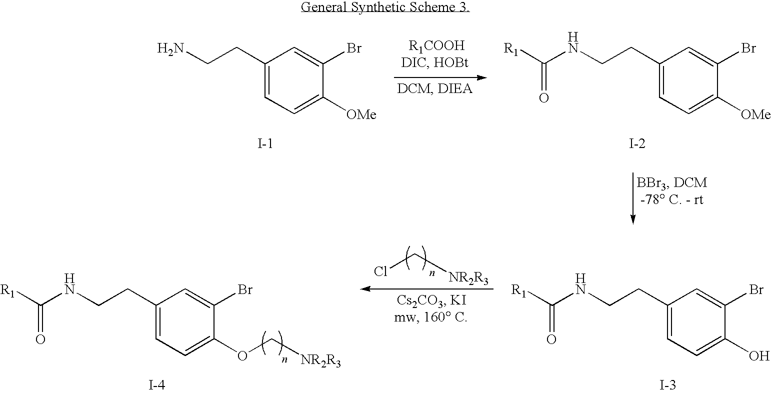

3. Coupling Step A

To a stirred solution of acid R₁COOH (1 equivalent), HOBt (2.1 equivalents), and amine I-1 (1 equivalent) in 9:1 CH₂Cl₂:DIEA at 25° C. was added DIC (2 equivalents) and the mixture was stirred overnight. After quenching with water, the reaction was added to a separatory funnel and washed 3 times with CH₂Cl₂. The organic layers were combined, and washed with saturated aqueous brine solution. The organic layer was dried over MgSO₄, and concentrated in vacuo to yield I-2. The crude material was then subjected to flash chromatography to give pure I-2 as a white solid (79-93% yield).

### N-(3-bromo-4-methoxyphenethyl)-1H-pyrrole-2-carboxamide

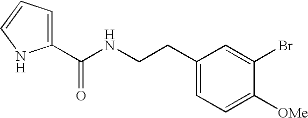

¹H NMR (400 MHz, DMSO-d₆) δ 9.31 (br s, 1H), 7.42 (d, J=2.0, 1H), 7.12 (dd, J=2.0, 8.4 Hz, 1H), 6.92 (m, 1H), 6.84 (d, J=8.4 Hz, 1H), 6.44 (s, 1H), 6.22 (m, 1H), 5.85 (m, 1H), 3.89 (s, 3H), 3.63 (q, J=6.8 Hz, 2H), 2.82 (t, J=7.2 Hz, 2H). ¹³C NMR (100 MHz, DMSO-d₆) δ 161.4, 154.5, 133.4, 231.4, 128.7, 125.7, 121.8, 112.0, 111.6, 109.0, 77.3, 76.9, 76.7, 56.2, 40.5, 34.7. HRMS (Q-TOF): m/z calc for C₁₄H₁₅BrN₂O₂ [M+H]: 323.0395; found 323.0408. 84.7% yield.

### 4-bromo-N-(3-bromo-4-methoxyphenethyl)thiophene-2-carboxamide

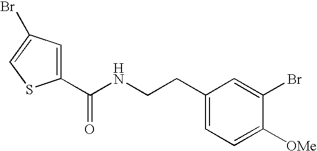

¹H NMR (400 MHz, DMSO-d₆) δ 7.41 (d, J=2.0 Hz, 1H), 7.37 (s, 1H), 7.27 (s, 1H), 7.12 (dd, J=2.0, 8.4 Hz, 1H), 6.86 (d, J=8.0 Hz, 1H), 5.93 (m, 1H), 3.89 (s, 3H), 3.63 (q, J=6.8 Hz, 2H), 2.84 (t, J=7.2 Hz, 2H), 2.17 (s, 1H). ¹³C NMR (100 MHz, DMSO-d₆) δ 159.8, 153.8, 141.1, 133.0, 132.9, 129.7, 129.1, 128.5, 112.5, 110.3, 108.6, 56.1, 40.7, 33.5.

HRMS (Q-TOF): m/z calc for C₁₄H₁₃Br₂NO₂S [M+H]: 417.9112; found 417.9120.79% yield.

### 5-bromo-N-(3-bromo-4-methoxyphenethyl)furan-2-carboxamide

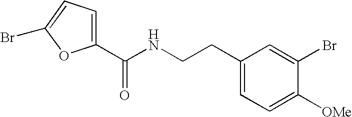

¹H NMR (400 MHz, DMSO-d₆) δ 7.42 (d, J=2.0 Hz, 1H), 7.13 (dd, J=2.0, 8.0 Hz, 1H), 7.06 (d, J=3.6 Hz, 1H), 6.85 (d, J=8.4 Hz, 1H), 6.43 (d, J=3.2 Hz, 1H), 6.32 (br s, 1H), 3.88 (s, 3H), 3.62 (m, 2H), 2.83 (t, J=7.2 Hz, 2H). ¹³C NMR (100 MHz, DMSO-d₆) δ 156.6, 153.8, 149.7, 133.0, 132.9, 129.1, 124.2, 115.6, 113.9, 112.5, 110.3, 56.1, 40.0, 33.6.

HRMS (Q-TOF): m/z calc for C₁₄H₁₃Br₂NO₃ [M+H]: 401.9340; found 401.9350.91% yield.

### N-(3-bromo-4-methoxyphenethyl)-1-methyl-1H-pyrrole-2-carboxamide

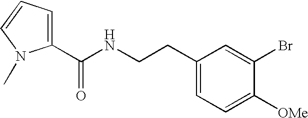

¹H NMR (400 MHz, DMSO-d₆) δ 8.01 (t, J=5.2 Hz, 1H), 7.43 (d, J=1.2 Hz, 1H), 7.18 (d, J=6.8 Hz, 1H), 7.01 (d, J=8.4 Hz, 1H), 6.86 (s, 1H), 6.70 (d, J=2.0 Hz, 1H), 5.98 (t, J=3.2 Hz, 1H), 3.81 (d, J=3.2 Hz, 6H), 3.36 (m, 2H). ¹³C NMR (100 MHz, DMSO-d₆) δ 161.3, 153.7, 133.45, 132.9, 129.1, 127.5, 125.7, 112.5, 112.5, 111.9, 110.3, 106.5, 56.1, 40.0, 36.0, 33.9. HRMS (Q-TOF): m/z calc for C₁₅H₁₇BrN₂O₂ [M+H]: 337.0552; found 337.0563.82% yield.

4. Deprotection Step A

To a stirred solution of coupled material I-2 (1 equivalent) in anhydrous CH₂Cl₂ under argon at −78° C. was added BBr₃ (4 equivalents of 1.0 M solution in CH₂Cl₂) over 20 minutes. The solution was stirred at −78° C. for 30 minutes and then allowed to warm to 25° C. for 1.5 hours. The reaction was slowly quenched with saturated aqueous NaHCO₃ until slightly basic by pH paper. This solution was added to a separatory funnel containing water and extracted 3× with CH₂Cl₂. The combined organic layers were washed with saturated aqueous brine solution. The organic layer was dried over MgSO₄, and concentrated in vacuo to yield the deprotected product I-3 (82-92% yield). This material was used without further purification.

### N-(3-bromo-4-hydroxyphenethyl)-1H-pyrrole-2-carboxamide

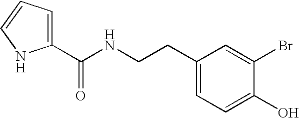

¹H NMR (400 MHz, DMSO-d₆) δ 11.36 (s, 1H), 7.99 (br s, 1H), 7.32, (d, J=2.0 Hz, 1H), 7.10 (dd, J=2.0, 8.0 Hz, 1H), 6.84 (d, J=8.4 Hz, 1H), 6.81 (br s, 1H), 6.70 (br s, 1H), 6.04 (m, 1H), 3.34 (q, J=6.8 Hz, 2H), 2.68 (t, J=7.2 Hz, 2H). ¹³C NMR (100 MHz, DMSO-d₆) δ 160.5, 152.2, 132.7, 131.8, 128.8, 126.3, 121.1, 116.2, 109.6, 108.9, 108.4, 40.2, 39.9, 39.7, 39.5, 39.3, 39.1, 34.0. HRMS (Q-TOF): m/z calc for C₁₃H₁₃BrN₂O₂ [M+H]: 309.0239; found 309.0241.92% yield.

### 4-bromo-N-(3-bromo-4-hydroxyphenethyl)thiophene-2-carboxamide

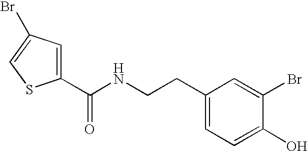

¹H NMR (400 MHz, DMSO-d₆) δ 9.99 (br s, 1H) 8.62 (t, J=5.6 Hz, 1H), 7.87 (d, J=1.2 Hz, 1H), 7.73 (d, J=1.2 Hz, 1H), 7.32 (d, J=1.6 Hz, 1H), 7.00 (dd, J=2.0, 8.4 Hz, 1H), 6.84 (d, J=8.4, 1H), 3.38 (q, J=6.8 Hz, 3H), 2.69 (t, J=7.2, 2H). ¹³C NMR (100 MHz, DMSO-d₆) δ 159.7, 152.3, 141.2, 132.7, 131.4, 129.7, 128.9, 128.5, 116.2, 109.0, 108.1, 40.8, 33.58. HRMS (Q-TOF): m/z calc for C₁₃H₁₁Br₂NO₂S [M+H]: 403.8955; found 403.8967. 82.2% yield.

### 5-bromo-N-(3-bromo-4-hydroxyphenethyl)furan-2-carboxamide

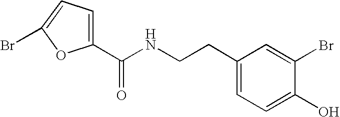

¹H NMR (400 MHz, DMSO-d₆) δ 9.98 (br s, 1H), 8.44 (t, J=5.6 Hz, 1H), 7.30 (d, J=1.6 Hz, 1H), 7.08 (d, J=3.6 Hz, 1H), 7.00 (dd, J=2.0, 8.4 Hz, 1H), 6.84 (d, J=8.4 Hz, 1H), 6.72 (d, J=3.6 Hz, 1H), 3.35 (q, J=6.8 Hz, 2H), 2.68 (t, J=7.2 Hz, 2H). ¹³C NMR (100 MHz, DMSO-d₆) δ 156.4, 152.1, 149.6, 132.5, 131.2, 128.7, 124.0, 116.0, 115.4, 113.7, 108.8, 39.9, 33.5. HRMS (Q-TOF): m/z calc for C₁₃H₁₁Br₂NO₃ [M+H]: 387.9184; found 387.9198. 84.2% yield.

### N-(3-bromo-4-hydroxyphenethyl)-1-methyl-1H-pyrrole-2-carboxamide

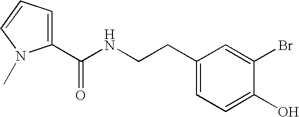

¹H NMR (400 MHz, DMSO-d₆) δ 9.97 (s, 1H), 7.98 (t, J=5.6 Hz, 1H), 7.31 (d, J=1.6 Hz, 1H), 7.00 (dd, J=1.6, 8.0 Hz, 1H), 6.85 (m, 2H), 6.68 (m, 1H), 5.97 (m, 1H), 3.79 (s, 3H), 3.31 (q, J=7.6 Hz, 2H), 2.69 (t, J=7.6 Hz, 2H). ¹³C NMR (100 MHz, DMSO-d₆) δ 161.7, 152.6, 133.1, 132.2, 129.3, 127.8, 126.1, 116.6, 112.3, 109.4, 106.9, 40.6, 36.4, 34.4.

HRMS (Q-TOF): m/z calc for C₁₄H₁₅BrN₂O₂ [M+H]: 323.0395; found 323.0398. 88.7% yield.

5. Alkylation Step A

In a 5 ml microwave vial containing I-3 (1 equivalent), alkyl halide (1.2 equivalents), KI (3 equivalents), and Cs₂CO₃ (3 equivalents) was added anhydrous DMF, 4 ml. This was heated under microwave conditions at 160° C. for 20-60 minutes. The reaction was filtered, concentrated in vacuo and purified via mass directed HPLC to obtain pure I-4 as the TFA salt (15-85% yield).

### 4-BROMO-N-(3-BROMO-4-(2-(PYRROLIDIN-1-YL)ETHOXY)PHENETHYL)-1H-PYRROLE-2-CARBOXAMIDE

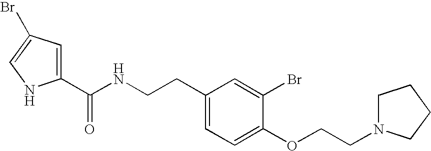

¹H NMR (400 MHz, DMSO-d₆) δ 10.06 (br s, 1H), 8.14 (t, J=5.2 Hz, 1H), 7.50 (d, J=2.0 Hz, 1H), 7.22 (dd, J=1.6, 8.4 Hz, 1H), 7.09 (d, J=8.4 Hz, 1H), 6.95 (m, 1H), 6.81 (m, 1H), 4.34 (t, J=4.4 Hz, 2H), 3.64 (m, 4H), 3.41 (q, J=6.4 Hz, 2H), 3.18 (m, 2H), 2.76 (t, J=6.8 Hz, 2H), 2.04 (m, 2H), 1.87 (m, 2H). ¹³C NMR (100 MHz, DMSO-d₆) δ 159.5, 152.3, 134.4, 133.1, 129.2, 126.9, 121.1, 113.8, 111.3, 110.6, 94.8, 64.8, 54.4, 52.8, 40.0, 33.7, 22.5. HRMS (Q-TOF): m/z calc for C₁₉H₂₃Br₂N₃O₂ [M+H]: 484.0235; found 484.0226.

### 4-BROMO-N-(3-BROMO-4-(2-(DIMETHYLAMINO)ETHOXY)PHENETHYL)-1H-PYRROLE-2-CARBOXAMIDE

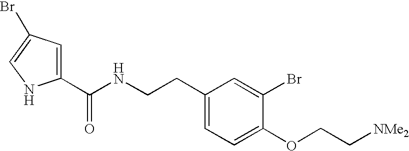

¹H NMR (400 MHz, DMSO-d₆) δ 9.98 (br s, 1H), 8.14 (t, J=4 Hz, 1H), 7.50 (d, J=2.0 Hz, 1H), 7.22 (dd, J=1.6, 8.4 Hz, 1H), 7.1 (d, J=8.4 Hz, 1H), 6.96 (m, 1H), 6.81 (m, 1H), 4.37 (t, J=4.8 Hz, 2H), 3.55 (m, 2H), 3.41 (q, J=6.4 Hz, 2H), 2.92 (s, 6H), 2.77 (m, 2H). ¹³C NMR (100 MHz, DMSO-d₆) δ 159.9, 152.6, 134.5, 133.3, 129.4, 127.9, 121.2, 114.0, 111.4, 110.8, 95.0, 63.1, 55.6, 43.5, 40.4, 33.9. HRMS (Q-TOF): m/z calc for C-₁₇H₂₁Br₂N₃O₂ [M+H]: 458.0079; found 458.0076.

### 4-bromo-N-(3-bromo-4-((1-methylpiperidin-3-yl)methoxy)phenethyl)-1H-pyrrole-2-carboxamide

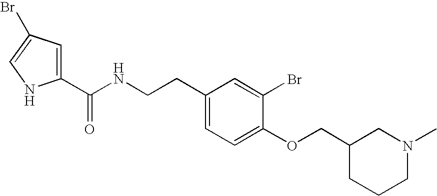

¹H NMR (400 MHz, DMSO-d₆) δ 9.71 (br s, 1H), 8.14 (t, J=5.2 Hz, 1H), 7.46 (d, J=2.0 Hz, 1H), 7.18 (dd, J=1.6, 8.4 Hz, 1H), 7.03 (d, J=8.4 Hz, 1H), 6.95 (m, 1H), 6.81 (m, 1H), 3.99 (m, 1H), 3.89 (m, 1H), 3.51 (m, 1H), 3.40 (m, 3H), 2.80 (m, 4H), 2.74 (t, J=6.8 Hz, 2H), 2.25 (m, 1H), 1.88 (m, 2H), 1.68 (m, 2H), 1.30 (m, 1H). ¹³C NMR (100 MHz, DMSO-d₆) δ 159.5, 152.7, 133.9, 132.9, 129.2, 126.9, 121.1, 113.9, 111.3, 110.9, 94.8, 70.3, 55.7, 53.7, 43.1, 40.0, 34.3, 33.7, 23.9, 22.1. HRMS (Q-TOF): m/z calc for C₂₀H₂₅Br₂N₃O₂ [M+H]: 498.0392; found 498.0407.

### N-(3-BROMO-4-(3-(DIMETHYLAMINO)PROPOXY)PHENETHYL)-1H-PYRROLE-2-CARBOXAMIDE

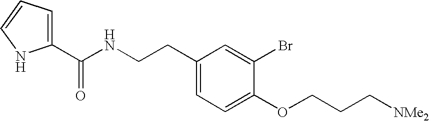

¹H NMR (400 MHz, DMSO-d₆) δ 9.72 (br s, 1H), 8.03 (t, J=5.2 Hz, 1H), 7.46 (d, J=2.0 Hz, 1H), 7.20 (dd, J=1.6, 8.4 Hz, 1H), 7.03 (d, J=8.4 Hz, 1H), 6.82 (s, 1H), 6.72 (s, 1H), 6.05 (m, 1H), 4.07 (t, J=6.0 Hz, 2H), 3.40 (q, J=6.4 Hz, 2H), 3.23 (m, 2H), 3.10 (s, 1H), 2.82 (s, 6H), 2.76 (t, J=6.8 Hz, 2H), 2.12 (m, 2H). ¹³C NMR (100 MHz, DMSO-d₆) δ 160.6, 152.7, 133.9, 132.9, 129.1, 126.3, 121.1, 113.8, 110.9, 109.7, 108.4, 66.0, 54.3, 42.3, 40.0, 33.9, 23.8. HRMS (Q-TOF): m/z calc for C₁₈H₂₄BrN₃O₂ [M+H]: 394.1130; found 394.1121.

### N-(3-bromo-4-(2-(pyrrolidin-1-yl)ethoxy)phenethyl)-1H-pyrrole-2-carboxamide

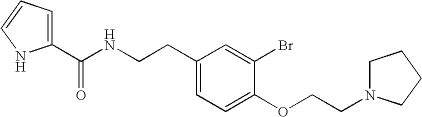

¹H NMR (400 MHz, DMSO-d₆) δ 10.10 (br s, 1H), 8.03 (t, J=4.4 Hz, 1H), 7.50 (d, J=2.0 Hz, 1H), 7.22 (dd, J=1.6, 8.4 Hz, 1H), 7.09 (d, J=8.4 Hz, 1H), 6.82 (s, 1H), 6.72 (s, 1H), 6.06 (m, 1H), 4.34 (t, J=4.4 Hz, 2H), 3.64 (m, 4H), 3.41 (q, J=6.0 Hz, 2H), 3.19 (m, 2H), 2.77 (t, J=7.2 Hz, 2H), 2.04 (m, 2H), 1.86 (m, 2H). ¹³C NMR (100 MHz, DMSO-d₆) δ 160.6, 152.3, 134.5, 133.1, 129.2, 126.3, 121.1, 113.8, 110.7, 109.7, 108.5, 64.8, 54.4, 52.8, 39.9, 33.9, 22.5. HRMS (Q-TOF): m/z calc for C₁₉H₂₄BrN₃O₂ [M+H]: 406.1130; found 406.1123.

### N-(3-bromo-4-(2-(dimethylamino)ethoxy)phenethyl)-1H-pyrrole-2-carboxamide

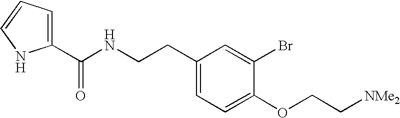

¹H NMR (400 MHz, DMSO-d₆) δ 10.10 (br s, 1H), 8.04 (t, J=5.2 Hz, 1H), 7.50 (d, J=2.0 Hz, 1H), 7.22 (dd, J=1.6, 8.4 Hz, 1H), 7.09 (d, J=8.4 Hz, 1H), 6.82 (s, 1H), 6.72 (s, 1H), 6.06 (m, 1H), 4.38 (t, J=4.8 Hz, 2H), 3.55 (m, 2H), 3.41 (q, J=6.0 Hz, 2H), 3.21 (s, 1H), 2.92 (s, 6H), 2.77 (t, J=6.0 Hz, 2H). ¹³C NMR (100 MHz, DMSO-d₆) δ 160.6, 152.2, 134.5, 133.1, 129.2, 126.3, 121.1, 113.8, 110.7, 109.7, 108.5, 63.9, 55.5, 43.3, 39.9, 33.9.

HRMS (Q-TOF): m/z calc for C₁₇H₂₂BrN₃O₂ [M+H]: 380.0974; found 380.0989.

### N-(3-bromo-4-((1-methylpiperidin-3-yl)methoxy)phenethyl)-1H-pyrrole-2-carboxamide

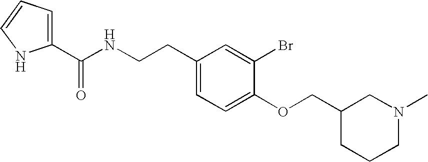

¹H NMR (400 MHz, DMSO-d₆) δ 9.90 (br s, 1H), 8.04 (t, J=5.2 Hz, 1H), 7.46 (d, J=2.0 Hz, 1H), 7.18 (dd, J=1.6, 8.4 Hz, 1H), 7.02 (d, J=8.4 Hz, 1H), 6.82 (s, 1H), 6.73 (s, 1H), 6.06 (m, 1H), 3.97 (m, 1H), 3.89 (m, 1H), 3.55 (m, 1H), 3.40 (m, 3H), 2.81 (m, 4H), 2.75 (t, J=6.8 Hz, 2H), 2.37 (m, 1H), 1.88 (m, 2H), 1.74 (m, 2H), 1.37 (m, 1H). ¹³C NMR (100 MHz, DMSO-d₆) δ 160.6, 152.7, 134.0, 132.9, 129.1, 126.3, 121.1, 113.9, 110.9, 109.8, 108.5, 70.3, 55.7, 53.6, 43.1, 40.0, 34.2, 34.0, 23.9, 22.1. HRMS (Q-TOF): m/z calc for C-₂₀H₂₆BrN₃O₂ [M+H]: 420.1287; found 420.1303.

### N-(3-bromo-4-(3-(dimethylamino)propoxy)phenethyl)-1-methyl-1H-pyrrole-2-carboxamide

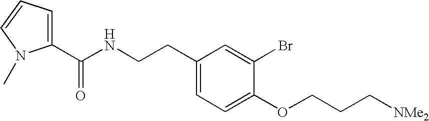

¹H NMR (400 MHz, DMSO-d₆) δ 9.56 (br s, 1H), 8.00 (t, J=5.6 Hz, 1H), 7.46 (d, J=2.0 Hz, 1H), 7.20 (dd, J=2.0, 8.4 Hz, 1H), 7.05 (d, J=8.4 Hz, 1H), 6.86 (s, 1H), 6.69 (m, 1H), 5.98 (t, J=2.4 Hz, 1H), 4.08 (t, J=6.0 Hz, 2H), 3.80 (s, 3H), 3.35 (q, J=6.4 Hz, 2H), 3.22 (m, 2H), 3.82 (s, 6H), 2.74 (t, J=7.2 Hz, 2H), 2.12 (m, 2H). ¹³C NMR (100 MHz, DMSO-d₆) δ 161.3, 152.7, 134.0, 132.9, 129.1, 127.5, 125.6, 113.8, 111.9, 110.8, 106.5, 66.0, 54.4, 42.4, 36.0, 33.9, 23.8. HRMS (Q-TOF): m/z calc for C₁₉H₂₆BrN₃O₂ [M+H]: 408.1287; found 408.1292.

### N-(3-bromo-4-(2-(pyrrolidin-1-yl)ethoxy)phenethyl)-1-methyl-1H-pyrrole-2-carboxamide

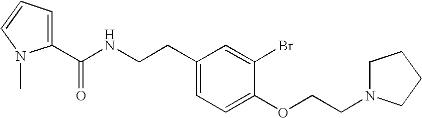

¹H NMR (400 MHz, DMSO-d₆) δ 10.27 (br s, 1H), 8.03 (t, J=5.6 Hz, 1H), 7.49 (d, J=2.0 Hz, 1H), 7.22 (dd, J=2.0, 8.4 Hz, 1H), 7.09 (d, J=8.4 Hz, 1H), 6.86 (s, 1H), 6.70 (m, 1H), 5.99 (t, J=2.8 Hz, 1H), 4.35 (t, J=4.8 Hz, 2H), 3.80 (s, 3H), 3.65 (m, 4H), 3.37 (q, J=6.8 Hz, 2H), 3.20 (m, 2H), 2.76 (t, J=6.8 Hz, 2H), 2.04 (m, 2H), 1.89 (m, 2H). ¹³C NMR (100 MHz, DMSO-d₆) δ 161.3, 152.3, 134.5, 133.1, 129.2, 127.5, 125.7, 113.8, 112.0, 110.6, 106.5, 64.8, 54.4, 52.8, 40.0, 36.0, 33.9, 22.5. HRMS (Q-TOF): m/z calc for C₂₀H₂₆BrN₃O₂ [M+H]: 420.1287; found 420.1272.

### N-(3-BROMO-4-(2-(DIMETHYLAMINO)ETHOXY)PHENETHYL)-1-METHYL-1H-PYRROLE-2-CARBOXAMIDE

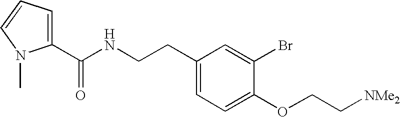

¹H NMR (400 MHz, DMSO-d₆) δ 10.17 (br s, 1H), 8.03 (t, J=5.6 Hz, 1H), 7.49 (d, J=2.9 Hz, 1H), 7.23, (dd, J=2.0, 8.4 Hz, 1H), 7.11 (d, J=8.4 Hz, 1H), 6.86 (s, 1H), 6.70 (m, 1H), 5.99 (t, J=2.8 Hz, 1H), 4.38 (t, J=5.2 Hz, 2H), 3.80 (s, 3H), 3.56 (m, 2H), 3.37 (q, J=6.8 Hz, 2H), 2.93 (s, 6H), 2.76 (t, J=7.2 Hz, 2H). ¹³C NMR (100 MHz, DMSO-d₆) δ 161.3, 152.2, 134.6, 133.1, 129.2, 127.5, 125.7, 113.9, 112.0, 110.7, 106.5, 64.0, 55.5, 43.3, 40.0, 36.0, 33.9. HRMS (Q-TOF): m/z calc for C₁₈H₂₄BrN₃O₂ [M+H]: 394.1130; found 394.1133.

### N-(3-bromo-4-((1-methylpiperidin-3-yl)methoxy)phenethyl)-1-methyl-1H-pyrrole-2-carboxamide

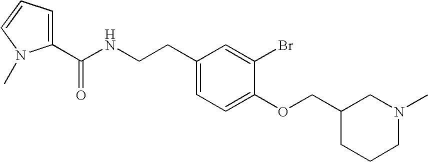

¹H NMR (400 MHz, DMSO-d₆) δ 9.89 (br s, 1H), 8.02 (t, J=5.2 Hz, 1H), 7.45 (d, J=2.0 Hz, 1H), 7.19 (dd, J=2.0, 8.4 Hz, 1H), 7.03 (d, J=8.4 Hz, 1H), 6.86 (s, 1H), 6.70 (m, 1H), 5.99 (t, J=2.8 Hz, 1H), 4.00 (m, 1H), 3.89 (m, 1H), 3.80 (s, 3H), 3.52 (m, 1H), 3.44 (m, 1H), 3.35 (q, J=6.8 Hz, 2H), 2.82 (m, 4H), 2.74 (t, J=7.2 Hz, 2H), 2.27 (m, 1H), 1.89 (m, 2H), 1.68 (m, 2H), 1.29 (m, 1H). ¹³C NMR (100 MHz, DMSO-d₆) δ 161.3, 152.7, 134.1, 132.9, 129.1, 127.5, 125.7, 113.9, 112.0, 110.9, 106.5, 70.3, 55.7, 53.6, 43.1, 40.0, 36.0, 34.2, 33.9, 23.9, 22.1. HRMS (Q-TOF): m/z calc for C₂₁H₂₈BrN₃O₂ [M+H]: 434.1443; found 434.1458.

### 4-BROMO-N-(3-BROMO-4-(3-(DIMETHYLAMINO)PROPOXY)PHENETHYL)THIOPHENE-2-CARBOXAMIDE

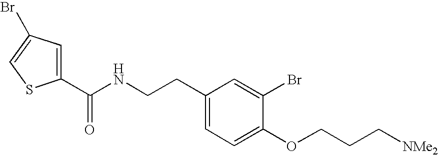

¹H NMR (400 MHz, DMSO-d₆) δ 9.50 (br s, 1H), 8.65 (t, J=5.6 Hz, 1H), 7.89 (d, J=1.2 Hz, 1H), 7.75 (d, J=1.2 Hz, 1H), 7.47 (d, J=2.0 Hz, 1H), 7.20 (dd, J=1.6, 8.4 Hz, 1H), 7.04 (d, J=8.4 Hz, 1H), 4.09 (t, J=6.0 Hz, 2H), 3.43 (q, J=6.0 Hz, 2H), 3.23 (m, 2H), 3.10 (s, 1H), 2.82 (s, 6H), 2.77 (t, J=7.2 Hz, 2H), 2.12 (m, 2H). ¹³C NMR (100 MHz, DMSO-d₆) δ 159.8, 152.8, 141.1, 133.6, 132.9, 129.7, 129.1, 128.5, 113.8, 110.9, 108.6, 66.0, 54.4, 42.4, 40.6, 33.5, 23.8. HRMS (Q-TOF): m/z calc for C₁₈H₂₂Br₂N₂O₂S [M+H]: 488.9847; found 488.9845.

### 4-bromo-N-(3-bromo-4-(2-(pyrrolidin-1-yl)ethoxy)phenethyl)thiophene-2-carboxamide

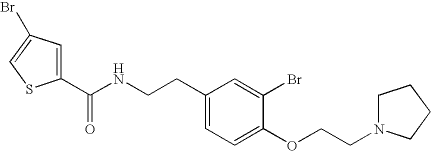

¹H NMR (400 MHz, DMSO-d₆) δ 10.08 (br s, 1H), 8.66 (t, J=5.6 Hz, 1H), 7.89 (d, J=1.6 Hz, 1H), 7.76 (d, J=1.6 Hz, 1H), 7.51 (d, J=2.0 Hz, 1H), 7.23 (dd, J=2.0, 8.4 Hz, 1H), 7.10 (d, J=8.4 Hz, 1H), 4.35 (t, J=4.8 Hz, 2H), 3.63 (m, 4H), 3.43 (q, J=6.8 Hz, 2H), 3.19 (m, 2H), 2.78 (t, J=7.2 Hz, 2H), 2.04 (m, 2H), 1.87 (m, 2H). ¹³C NMR (100 MHz, DMSO-d₆) δ 159.7, 152.3, 134.1, 133.2, 129.7, 129.2, 128.5, 113.8, 64.7, 54.4, 52.8, 40.6, 35.7, 33.8, 22.5. HRMS (Q-TOF): m/z calc for C₁₉H₂₂Br₂N₂O₂S [M+H]: 500.9847; found 500.9847.

### 4-BROMO-N-(3-BROMO-4-(2-(DIMETHYLAMINO)ETHOXY)PHENETHYL)THIOPHENE-2-CARBOXAMIDE

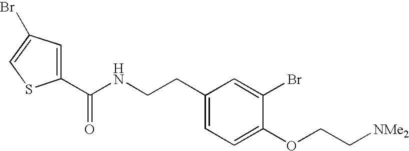

¹H NMR (400 MHz, DMSO-d₆) δ 9.85 (br s, 1H), 8.66 (t, J=5.2 Hz, 1H), 7.89 (d, J=0.8 Hz, 1H), 7.76 (d, J=0.8 Hz, 1H), 7.50 (d, J=1.6 Hz, 1H), 7.23 (dd, J=1.6, 8.4 Hz, 1H), 7.11 (d, J=8.4 Hz, 1H), 4.37 (t, J=4.8 Hz, 2H), 3.50 (m, 4H), 3.21 (s, 1H), 2.92 (s, 6H), 2.78 (t, J=7.2 Hz, 2H). ¹³C NMR (100 MHz, DMSO-d₆) δ 159.8, 152.3, 141.1, 134.2, 133.1, 129.7, 129.2, 128.5, 113.9, 110.7, 108.6, 63.9, 55.5, 43.4, 40.6, 33.5. HRMS (Q-TOF): m/z calc for C₁₇H₂₀Br₂N₂O₂S [M+H]: 474.9690; found 474.9705.

### BROMO-N-(3-BROMO-4-((1-METHYLPIPERIDIN-3-YL)METHOXY)PHENETHYL)THIOPHENE-2-CARBOXAMIDE

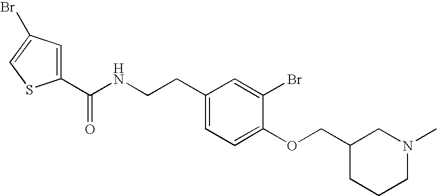

¹H NMR (400 MHz, DMSO-d₆) δ 9.62 (br s, 1H), 8.65 (t, J=5.6 Hz, 1H), 7.89 (d, J=1.2 Hz, 1H), 7.57 (d, J=1.2 Hz, 1H), 7.47 (d, J=1.6 Hz, 1H), 7.19 (dd, J=1.6, 8.4 Hz, 1H), 7.03 (d, J=8.4 Hz, 1H), 4.00 (m, 1H), 3.89 (m, 1H), 3.51 (m, 1H), 3.43 (m, 3H), 2.81 (m, 6H), 2.25 (m, 1H), 1.89 (m, 2H), 1.70 (m, 2H), 1.29 (m, 1H). ¹³C NMR (100 MHz, DMSO-d₆) δ 159.8, 152.7, 141.1, 133.6, 133.0, 129.7, 129.2, 128.5, 113.9, 110.9, 108.6, 70.3, 55.7, 53.7, 43.1, 40.6, 34.3, 33.5, 23.9, 22.1. HRMS (Q-TOF): m/z calc for C₂₀H₂₄Br₂N₂O₂S [M+H]: 515.0003; found 515.0017.

### 5-BROMO-N-(3-BROMO-4-(3-(DIMETHYLAMINO)PROPOXY)PHENETHYL)FURAN-2-CARBOXAMIDE

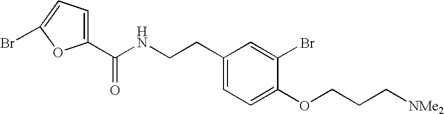

¹H NMR (400 MHz, DMSO-d₆) δ 9.52 (br s, 1H), 8.48 (t, J=5.6 Hz, 1H), 7.45 (d, J=2.0 Hz, 1H), 7.19 (dd, J=2.0, 8.4 Hz, 1H), 7.10 (d, J=3.6 Hz, 1H), 7.03 (d, J=8.4 Hz, 1H), 6.74 (d, J=3.6 Hz, 1H), 4.09 (t, J=6.0 Hz, 2H), 3.39 (q, J=6.8 Hz, 2H), 3.23 (m, 2H), 2.83 (s, 6H), 2.76 (t, J=7.2 Hz, 2H), 2.10 (m, 2H). ¹³C NMR (100 MHz, DMSO-d₆) δ 156.6, 152.8, 149.7, 133.6, 132.9, 129.1, 124.2, 115.6, 113.9, 110.9, 66.0, 54.4, 42.4, 33.5, 23.8.

HRMS (Q-TOF): m/z calc for C₁₈H₂₂Br₂N₂O₃ [M+H]: 473.0075; found 473.0067.

### 5-BROMO-N-(3-BROMO-4-(2-(PYRROLIDIN-1-YL)ETHOXY)PHENETHYL)FURAN-2-CARBOXAMIDE

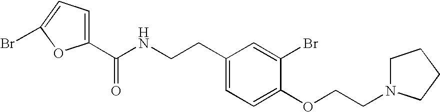

¹H NMR (400 MHz, DMSO-d₆) δ 10.2, 8.50 (t, J=5.6 Hz, 1H), 7.48 (d, J=1.2 Hz, 1H), 7.21 (dd, J=1.6, 8.4 Hz, 1H), 7.10 (m, 2H), 6.73 (d, J=3.6 Hz, 1H), 4.35 (t, J=4.8 Hz, 2H), 3.64 (m, 4H), 3.40 (q, J=6.8 Hz, 2H), 3.24 (m, 2H), 2.77 (t, J=7.2 Hz, 2H), 2.04 (m, 2H), 1.89 (m, 2H). ¹³C NMR (100 MHz, DMSO-d₆) δ 156.6, 152.3, 149.7, 134.1, 133.1, 129.2, 124.2, 115.6, 113.9, 113.8, 110.6, 64.7, 54.4, 52.8, 39.9, 33.6, 22.5. HRMS (Q-TOF): m/z calc for C₁₉H₂₂Br₂N₂O₃ [M+H]: 485.0075; found 485.0087.

### 5-BROMO-N-(3-BROMO-4-(2-(DIMETHYLAMINO)ETHOXY)PHENETHYL)FURAN-2-CARBOXAMIDE

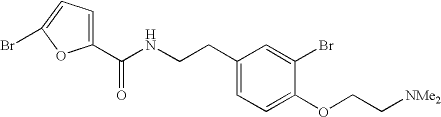

¹H NMR (400 MHz, DMSO-d₆) δ 10.08 (br s, 1H), 8.50 (t, J=5.6 Hz, 2H), 7.48 (d, J=1.6 Hz, 1H), 7.21 (dd, J=1.6, 8.4 Hz, 1H), 7.10 (m, 2H), 6.73 (d, J=3.6 Hz, 1H), 4.38 (t, J=4.8 Hz, 2H), 3.55 (m, 2H), 3.40 (q, J=6.8 Hz, 2H), 2.77 (t, J=6.8 Hz, 2H). ¹³C NMR (100 MHz, DMSO-d₆) δ 156.6, 152.3, 149.7, 134.2, 133.1, 129.2, 124.2, 115.6, 113.9, 113.8, 110.7, 63.9, 55.5, 43.3, 40.0, 33.6. HRMS (Q-TOF): m/z calc for C₁₇H₂₀Br₂N₂O₃ [M+H]: 458.9919; found 458.9916.

### 5-BROMO-N-(3-BROMO-4-((1-METHYLPIPERIDIN-3-YL)METHOXY)PHENETHYL)FURAN-2-CARBOXAMIDE

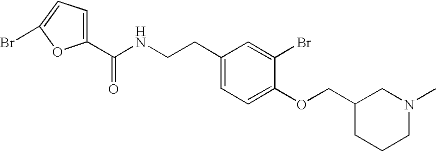

¹H NMR (400 MHz, DMSO-d₆) δ 9.58 (br s, 1H), 8.48 (t, J=5.6 Hz, 1H), 7.45 (d, J=2.0 Hz, 1H), 7.17 (dd, J=1.6, 8.4 Hz, 1H), 7.09 (d, J=3.6 Hz, 1H), 7.03 (d, J=8.4 Hz, 1H), 6.73 (d, J=3.6 Hz, 1H), 3.99 (m, 1H), 3.89 (m, 1H), 3.52 (m, 1H), 3.41 (m, 3H), 2.81 (m, 6H), 2.49 (m, 1H), 1.89 (m, 2H), 1.70 (m, 2H), 1.29 (m, 1H). ¹³C NMR (100 MHz, DMSO-d₆) δ 156.6, 152.7, 149.7, 133.7, 132.9, 129.1, 124.2, 115.6, 113.9, 113.8, 110.9, 70.3, 55.7, 53.6, 43.2, 34.3, 33.6, 23.9, 22.1. HRMS (Q-TOF): m/z calc for C₂₀H₂₄Br₂N₂O₃ [M+H]: 499.0232; found 499.0251.

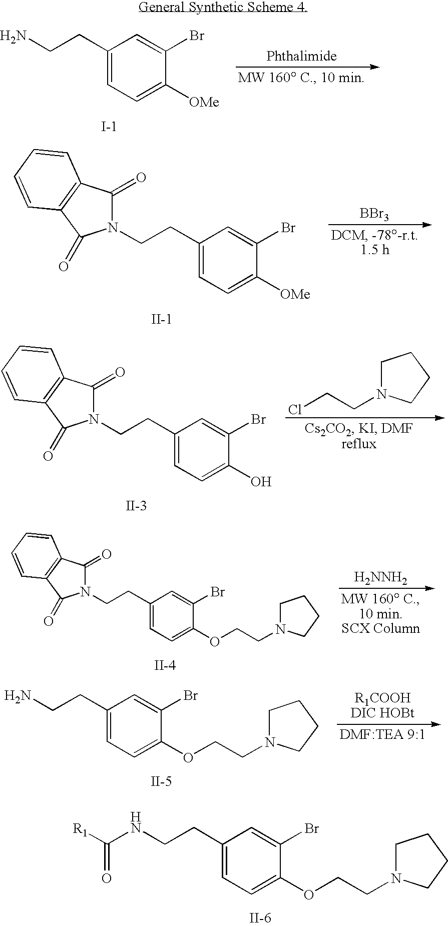

6. Phthalimide Formation Step

A 20 ml microwave vial containing I-1 (1 equivalent), and phthalic anhydride (1 equivalent), was capped and heated to 160° C. for 30 min. The solid was dissolved in hot EtOAc and left to crystallize overnight. The crystals were filtered and washed with ether to obtain II-1 as a white solid (94% yield).

### 2-(3-bromo-4-methoxyphenethyl)isoindoline-1,3-dione

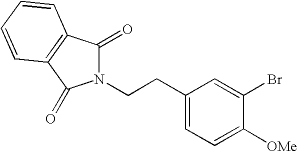

¹H NMR (400 MHz, DMSO-d₆) δ 7.83 (m, 4H), 7.41 (d, J=1.6 Hz, 1H), 7.13 (dd, J=8.4, 2.0 Hz, 1H), 6.96 (d, J=8.4 Hz, 1H), 3.78 (m, 5H), 2.86 (t, J=7.2 Hz, 2H). LCMS, single peak, 3.43 min, m/e, 360.0 (M+1)

7. Deprotection Step B

To a stirred solution of protected material II-1 (1 equivalent) in anhydrous CH₂Cl₂ under argon at −78° C. was added BBr₃ (4 equivalents of 1.0 M solution in CH₂Cl₂) over 20 minutes. The solution was stirred at −78° C. for 30 minutes and then allowed to warm to 25° C. for 1.5 hours. The reaction was slowly quenched with saturated aqueous NaHCO₃ until slightly basic by pH paper. This solution was added to a separatory funnel containing water and extracted 3× with CH₂Cl₂. The combined organic layers were washed with saturated aqueous brine solution. The organic layer was dried over MgSO₄, and concentrated in vacuo to yield the deprotected product II-3 (100% yield). This material was used without further purification.

### 2-(3-BROMO-4-HYDROXYPHENETHYL)ISOINDOLINE-1,3-DIONE

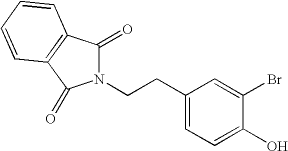

¹H NMR (400 MHz, DMSO-d₆) δ 10.03 (s, 1H), 7.83)m, 4H), 7.29 (d, J=1.6 Hz, 1H), 6.96 (dd, J=8.4, 2.0 Hz, 1H), 6.79 (d, J=8.0 Hz, 1H), 3.75 (t, J=7.2 Hz, 2H), 2.80 (t, J=7.2 Hz, 2H); LCMS, single peak, 2.98 min, m/e, 346.0 (M+1).

8. Alkylation Step B

In a 200 ml round bottom flask containing II-3 (1 equivalent), alkyl halide (2 equivalents), KI (2 equivalents), and Cs₂CO₃ (2 equivalents) was added anhydrous DMF, 30 ml which was heated to reflux overnight. The reaction was added to a seperatory funnel containing water and washed 2× with EtOAc. The combined organic layers were washed with saturated aqueous brine solution. The organic layer was dried over MgSO₄, and concentrated in vacuo to yield II-4. This material was used immediately without further purification.

9. Deprotection Step C

A 20 ml microwave vial containing II-4 (1 equivalent) ethanol, 10 ml, and hydrazine hydrate (10 equivalents), was capped and heated to 120° C. for 20 min. The solution was immediately added to a SCX cartridge and washed 5× with methanol. The cartidge was then washed 2× with 2M ammonia in methanol and concentrated in vacuo to yield II-5 (60% yield over 2 steps).

### 2-(3-bromo-4-(2-(pyrrolidin-1-yl)ethoxy)phenyl)ethanamine

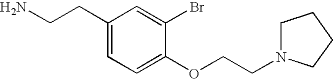

¹H NMR (400 MHz, DMSO-d₆) δ 7.38 (d, J=2.0 Hz, 1H), 7.12 (dd, J=8.4, 1.6 Hz, 1H), 6.99 (d, J=8.4 Hz, 1H), 4.08 (t, J=5.6 Hz, 2H), 2.78 (t, J=5.6 Hz, 2H), 2.71 (m, 4H), 2.55 (m, 6H), 1.65 (m, 4H); LCMS, single peak, 1.60 min, m/e, 313.1 (M+1).

10. Coupling Step B

To a stirred solution of acid R₁COOH (1 equivalent), HOBt (2.1 equivalents), and amine II-5 (1 equivalent) in 9:1 CH₂Cl₂:DIEA at 25° C. was added DIC (2 equivalents) and the mixture was stirred overnight. The reaction was filtered, concentrated in vacuo and purified via mass directed HPLC to obtain pure II-6 as the TFA salt (50-95% yield).

### N-(3-BROMO-4-(2-(PYRROLIDIN-1-YL)ETHOXY)PHENETHYL)THIAZOLE-5-CARBOXAMIDE

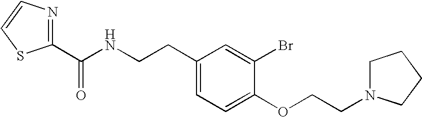

¹H NMR (400 MHz, CDCl₃) δ 7.84 (d, J=3.2 Hz, 1H), 7.57 (d, J=3.2 Hz, 1H), 7.44 (d, J=2.0 Hz, 1H), 7.15 (dd, J=8.4, 2.0 Hz, 1H), 6.83 (d, J=8.4 Hz, 1H), 4.41 (m, 2H), 3.95 (m, 2H), 3.67 (q, J=6.8 Hz, 2H), 3.15 (m, 2H), 2.87 (t, J=6.8 Hz, 2H), 2.15 (m, 6H); LCMS, single peak, 2.27 min, m/e, 424.1 (M+1).

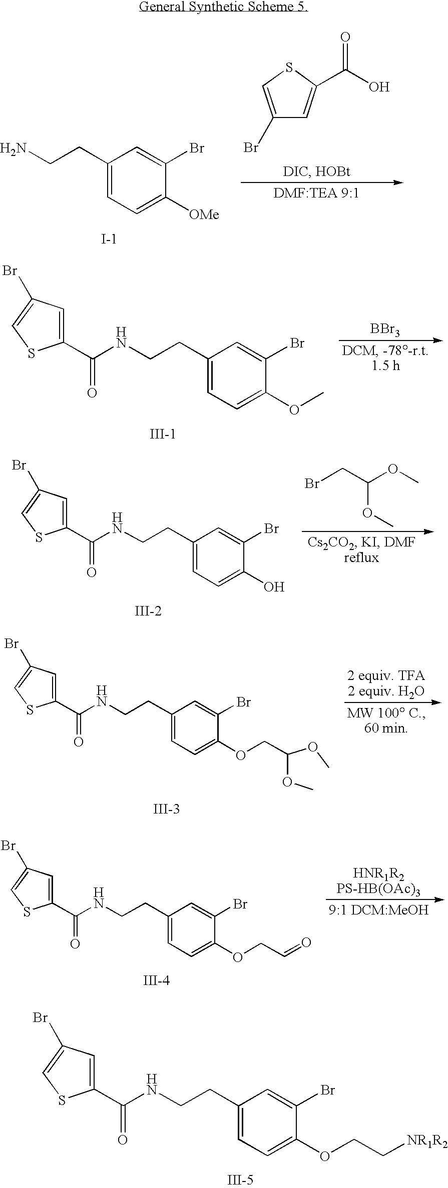

11. Coupling Step C

To a stirred solution of acid R₁COOH (1 equivalent), HOBt (2.1 equivalents), and amine I-1 (1 equivalent) in 9:1 CH₂Cl₂:DIEA at 25° C. was added DIC (2 equivalents) and the mixture was stirred overnight. After quenching with water, the reaction was added to a separatory funnel and washed 3× with CH₂Cl₂. The organic layers were combined, and washed with saturated aqueous brine solution. The organic layer was dried over MgSO₄, and concentrated in vacuo. The crude material was then subjected to flash chromatography to give pure III-1 as a white solid (93% yield).

### 4-BROMO-N-(3-BROMO-4-METHOXYPHENETHYL)THIOPHENE-2-CARBOXAMIDE

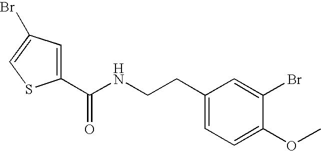

¹H NMR (400 MHz, DMSO-d₆) δ 7.41 (d, J=2.0 Hz, 1H), 7.37 (s, 1H), 7.27 (s, 1H), 7.12 (dd, J=2.0, 8.4 Hz, 1H), 6.86 (d, J=8.0 Hz, 1H), 5.93 (m, 1H), 3.89 (s, 3H), 3.63 (q, J=6.8 Hz, 2H), 2.84 (t, J=7.2 Hz, 2H), 2.17 (s, 1H). ¹³C NMR (100 MHz, DMSO-d₆) δ 159.8, 153.8, 141.1, 133.0, 132.9, 129.7, 129.1, 128.5, 112.5, 110.3, 108.6, 56.1, 40.7, 33.5.

HRMS (Q-TOF): m/z calc for C₁₄H₁₃Br₂NO₂S [M+H]: 417.9112; found 417.9120.79% yield.

12. Deprotection Step D

To a stirred solution of coupled material III-1 (1 equivalent) in anhydrous CH₂Cl₂ under argon at −78° C. was added BBr₃ (4 equivalents of 1.0 M solution in CH₂Cl₂) over 20 minutes. The solution was stirred at −78° C. for 30 minutes and then allowed to warm to 25° C. for 1.5 hours. The reaction was slowly quenched with saturated aqueous NaHCO₃ until slightly basic by pH paper. This solution was added to a separatory funnel containing water and extracted 3× with CH₂Cl₂. The combined organic layers were washed with saturated aqueous brine solution. The organic layer was dried over MgSO₄, and concentrated in vacuo to yield the deprotected product III-2 (92% yield). This material was used without further purification.

### 4-bromo-N-(3-bromo-4-hydroxyphenethyl)thiophene-2-carboxamide

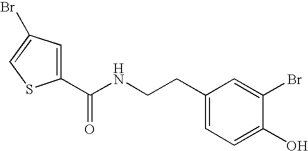

¹H NMR (400 MHz, DMSO-d₆) δ 9.99 (br s, 1H) 8.62 (t, J=5.6 Hz, 1H), 7.87 (d, J=1.2 Hz, 1H), 7.73 (d, J=1.2 Hz, 1H), 7.32 (d, J=1.6 Hz, 1H), 7.00 (dd, J=2.0, 8.4 Hz, 1H), 6.84 (d, J=8.4, 1H), 3.38 (q, J=6.8 Hz, 3H), 2.69 (t, J=7.2, 2H). ¹³C NMR (100 MHz, DMSO-d₆) δ 159.7, 152.3, 141.2, 132.7, 131.4, 129.7, 128.9, 128.5, 116.2, 109.0, 108.1, 40.8, 33.58. HRMS (Q-TOF): m/z calc for C₁₃H₁₁Br₂NO₂S [M+H]: 403.8955; found 403.8967. 82.2% yield.

13. Alkylation Step C

In a 20 ml microwave vial containing III-2 (1 equivalent), alkyl halide (4 equivalents), KI (2 equivalents), and Cs₂CO₃ (2 equivalents) was added anhydrous DMF, 10 ml. This was heated under microwave conditions at 120° C. for 60 minutes. The reaction was added to a seperatory funnel containing water and washed 3× with EtOAc. The combined organic layers were washed with saturated aqueous brine solution. The organic layer was dried with MgSO₄ concentrated in vacuo. This was filtered through a silica plug, washed 3× with EtOAc, and concentrated in vacuo to yield III-3 (100%). This material was used without further purification.

### 4-BROMO-N-(3-BROMO-4-(2,2-DIMETHOXYETHOXY)PHENETHYL)THIOPHENE-2-CARBOXAMIDE

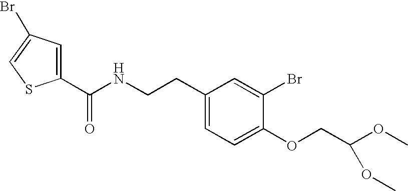

¹H NMR (400 MHz, DMSO-d₆) δ 8.64 (t, J=5.2 Hz, 1H), 7.88 (s, 1H), 7.75 (s, 1H), 7.45 (d, J=2.0 Hz, 1H), 7.16 (dd, J=8.4, 1.6 Hz, 1H), 7.06 (d, J=8.4, 1H), 4.68 (t, J=5.2 Hz, 1H), 4.00 (d, J=7.2 Hz, 2H), 3.42 (q, J=6.0 Hz, 2H), 3.37 (s, 6H), 2.76 (t, J=6.8 Hz, 2H).

14. Aldehyde Generation

A 20 ml microwave vial containing III-3 (1 equivalent) 1,2 dichloroethane, 10 ml, TFA (2 equivalents), and water (2 equivalents), was capped and heated to 100° C. for 60 min. This was concentrated in vacuo and used without any further purification.

15. Reductive Amination

To a 20 ml vial containing III-4 and 9:1 DCM:MeOH, 10 ml, was added PS-triacetoxyborohydride (5 equivalents), and amine (5 equivalents), which was agitated overnight. The reaction was concentrated in vacuo and purified via mass directed HPLC to obtain pure III-6 as the TFA salt (15-70% yield).

### (R)-4-bromo-N-(3-bromo-4-(2-(2-methylpyrrolidin-1-yl)ethoxy)phenethyl)thiophene-2-carboxamide

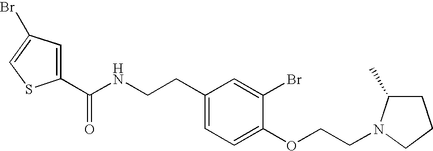

¹H NMR (400 MHz, DMSO-d₆) δ 9.86 (br s, 1H), 8.70 (t, J=5.6 Hz, 1H), 7.88 (s, 1H), 7.77 (s, 1H), 7.50 (d, J=2.0 Hz, 1H), 7.23 (dd, J=8.4, 2.0 Hz, 1H), 7.07 (d, J=8.4 Hz, 1H), 4.35 (m, 2H), 3.75 (m, 2H), 3.52 (m, 2H), 3.43 (q, J=6.8 Hz, 2H), 3.33 (m, 1H), 2.78 (t, J=7.2 Hz, 2H), 2.21 (m, 1H), 1.94 (m, 2H), 1.60 (m, 1H), 1.38 (d, J=6.4 Hz, 3H); LCMS, single peak, 2.65 min, m/e, 515.0 (M+1).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A synthetic compound comprising a structure represented by a formula: 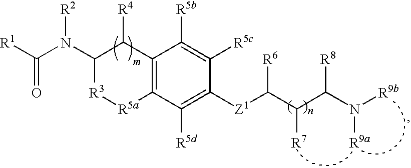
wherein R¹ is selected from optionally substituted aryl, heteroaryl, cycloalkyl, heterocycloalkyl, cycloalkenyl, or heterocycloalkenyl;
wherein R² is selected from hydrogen, an optionally substituted organic residue comprising from 1 to 6 carbons, or a hydrolysable residue;
wherein each of R³, R⁴ (if present), R⁶, R⁷ (if present), and R⁸ independently comprises two residues independently selected from hydrogen and an optionally substituted organic residue comprising from 1 to 6 carbons;
wherein Z¹ is O, S, or NR¹⁰, wherein R¹⁰, when present, is hydrogen or an optionally substituted organic residue comprising from 1 to 12 carbons;
wherein each ---- is, independently, an optional covalent bond;
wherein m and n are, independently, integers selected from 0, 1, and 2;
wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and
wherein each of R^{9a} and R^{9b} independently comprises hydrogen or an optionally substituted organic residue comprising from 1 to 12 carbons;
or a pharmaceutically acceptable derivative or N-oxide thereof.

2. The compound of claim 1, wherein the compound is not Dispyrin, Purealidin Q, Purealidin S, Purpurealidin A, Purpurealidin B, or Fistularin-3.

3. The compound of claim 1, wherein R¹ comprises a structure represented by a formula: 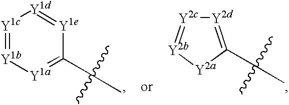
wherein each of Y^{1a}, Y^{1b}, Y^{1c}, Y^{1d}, and Y^{1e} is independently selected from nitrogen or CR¹¹, wherein each R¹¹, when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons;
with the proviso that no more than two of Y^{1a}, Y^{1b}, Y^{1c}, Y^{1d}, and Y^{1e} are nitrogen; and
wherein Y^{2a} is selected from O, S, and NR¹², wherein R¹², if present, is selected from hydrogen or an alkyl residue comprising from 1 to 4 carbons;
wherein each of Y^{2b}, Y^{2c}, and Y^{2d} is independently selected from N and CR¹², wherein each R¹², when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons;
with the proviso that no more than three of Y^{2a}, Y^{2b}, Y^{2c}, and Y^{2d} are heteroatoms.

4. The compound of claim 1, wherein R¹ is selected from optionally substituted pyridyl, optionally substituted oxazolyl, optionally substituted triazolyl, optionally substituted thiazolyl, optionally substituted aryl, optionally substituted thiopheneyl, optionally substituted pyrrolyl, optionally substituted alkyl pyrrolyl, and optionally substituted furanyl.

5. The compound of claim 1, wherein R¹ is selected from:

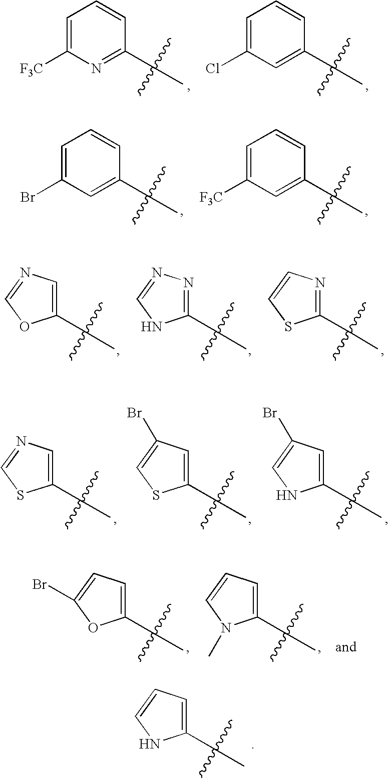

6. The compound of claim 1, wherein R² is hydrogen.

7. The compound of claim 1, wherein each of R^{5a} and R^{5b} independently comprises hydrogen, and wherein one of R^{5c} and R^{5d} is halogen, and one of R^{5c} and R^{5d} is hydrogen.

8. The compound of claim 1, wherein the compound comprises a structure represented by the formula: 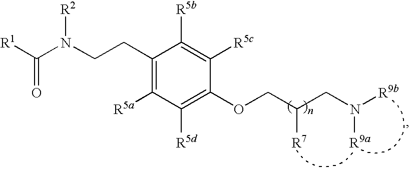
wherein at least one of R^{5c} and R^{5d} is halogen.

9. The compound of claim 1, wherein the compound comprises a structure represented by a formula: 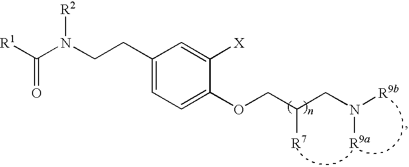
wherein X is F, Cl, Br, or I.
wherein n is an integer from 0 to 1.

10. The compound of claim 1, wherein the compound comprises a structure represented by a formula selected from: 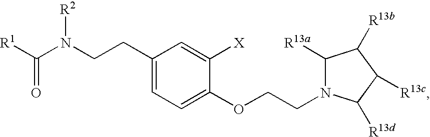
wherein X is F, Cl, Br, or I; and
wherein each of R^{13a}, R^{13b}, R^{13c}, and R^{13d} is independently selected from hydrogen, alkyl comprising from 1 to 4 carbons, and halide; or 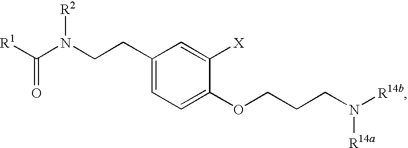
wherein X is F, Cl, Br, or I; and
wherein each of R^{14a} and R^{14b} is independently selected from hydrogen and alkyl comprising from 1 to 4 carbons; or 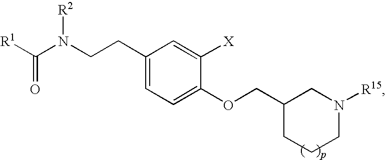
wherein X is F, Cl, Br, or I;
wherein R¹⁵ comprises hydrogen or alkyl comprising from 1 to 4 carbons; and
wherein p is an integer from 0-2.

11. A method of preparing a compound comprising the step of reacting a compound comprising a structure represented by a formula: 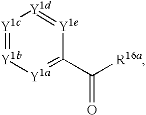
wherein each of Y^{1a}, Y^{1b}, Y^{1c}, Y^{1d}, and Y^{1e} is independently selected from nitrogen or CR¹¹, wherein each R¹¹, when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons;
wherein R^{16a} comprises OH, alkoxy, acyloxy, hydrogen, or halogen;
with the proviso that no more than two of Y^{1a}, Y^{1b}, Y^{1c}, Y^{1d}, and Y^{1e} are nitrogen; or 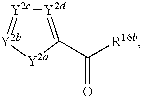
wherein Y^{2a} is selected from O, S, and NR¹², wherein R¹², if present, is selected from hydrogen or an alkyl residue comprising from 1 to 4 carbons;
wherein each of Y^{2b}, Y^{2c}, and Y^{2d} is independently selected from N and CR¹², wherein each R¹², when present, is independently selected from hydrogen, halide, trifluoromethyl, hydroxyl, amino, cyano, nitro, azide, carboxamido, alkoxy, thiol, and an optionally substituted organic residue comprising from 1 to 6 carbons;
wherein R^{16b} comprises OH, alkoxy, acyloxy, hydrogen, or halogen;
with the proviso that no more than three of Y^{2a}, Y^{2b}, Y^{2c}, and Y^{2d} are heteroatoms.
with a compound having a structure represented by a formula: 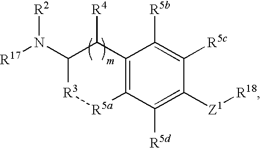
wherein R² is selected from hydrogen, an optionally substituted organic residue comprising from 1 to 6 carbons, or a hydrolysable residue;
wherein each of R³, and R⁴ (if present) independently comprises two residues independently selected from hydrogen and an optionally substituted organic residue comprising from 1 to 6 carbons;
wherein Z¹ is O, S, or NR¹⁰, wherein R¹⁰, when present, is hydrogen or an optionally substituted organic residue comprising from 1 to 12 carbons;
wherein ---- is an optional covalent bond;
wherein m an integers selected from 0, 1, and 2;
wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon;
wherein R¹⁷ is hydrogen or a hydrolyzable group;
wherein R¹⁸ is hydrogen, a hydrolyzable group, a protecting group, or an optionally substituted organic residue comprising from 1 to 12 carbons;
thereby forming an amide bond.

12. The method of claim 11, wherein R¹⁸ comprises a structure represented by a formula: 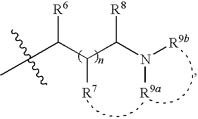
wherein n is an integer selected from 0, 1, and 2;
wherein R⁶, R⁷ (if present), and R⁸ independently comprises two residues independently selected from hydrogen and an optionally substituted organic residue comprising from 1 to 6 carbons; and
wherein each of R^{9a} and R^{9b} independently comprises hydrogen or an optionally substituted organic residue comprising from 1 to 12 carbons.

13. The method of claim 11, wherein R¹⁸ is hydrogen or a hydrolyzable group, further comprising the step of reacting with a compound having a structure represented by a formula: 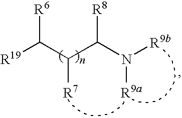
wherein n is an integer selected from 0, 1, and 2;
wherein R⁶, R⁷ (if present), and R⁸ independently comprises two residues independently selected from hydrogen and an optionally substituted organic residue comprising from 1 to 6 carbons;
wherein each of R^{9a} and R^{9b} independently comprises hydrogen or an optionally substituted organic residue comprising from 1 to 12 carbons; and
wherein R¹⁹ is a leaving group;
thereby forming an ether.

14. The method of claim 11, wherein the compound prepared is a compound of claim 1.

15. A method of modulating the activity of a G-protein coupled receptor in a subject in need thereof comprising the step of administering to the subject a therapeutically effective amount of at least one compound comprising a structure represented by a formula: 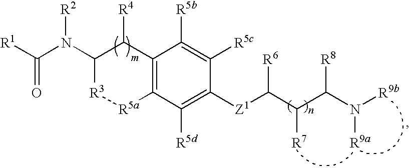
wherein R¹ is selected from optionally substituted aryl, heteroaryl, cycloalkyl, heterocycloalkyl, cycloalkenyl, or heterocycloalkenyl;
wherein R² is selected from hydrogen, an optionally substituted organic residue comprising from 1 to 6 carbons, or a hydrolysable residue;
wherein each of R³, R⁴ (if present), R⁶, R⁷ (if present), and R⁸ independently comprises two residues independently selected from hydrogen and an optionally substituted organic residue comprising from 1 to 6 carbons;
wherein Z¹ is O, S, or NR¹⁰, wherein R¹⁰, when present, is hydrogen or an optionally substituted organic residue comprising from 1 to 12 carbons;
wherein each ---- is, independently, an optional covalent bond;
wherein m and n are, independently, integers selected from 0, 1, and 2;
wherein each of R^{5a}, R^{5b}, R^{5c}, and R^{5d} is independently selected from hydrogen, halide, hydroxyl, trifluoromethyl, amino, cyano, nitro, azide, carboxamido, alkoxy, thiol, and an optionally substituted organic residue comprising from 1 to 6 carbon; and
wherein each of R^{9a} and R^{9b} independently comprises hydrogen or an optionally substituted organic residue comprising from 1 to 12 carbons;
or a pharmaceutically acceptable derivative or N-oxide thereof,
thereby modulating activity of the G-protein coupled receptor in the subject.

16. The method of claim 15, wherein the G-protein coupled receptor is an H3 receptor.

17. The method of claim 15, wherein modulating the activity of a G-protein coupled receptor in a subject treats a disorder associated with G-protein coupled receptor activity in the subject.

18. The method of claim 17, wherein the subject has been diagnosed with the disorder prior to the administering step.

19. The method of claim 17, further comprising the step of identifying a subject with the disorder.

20. The method of claim 17, wherein the disorder is selected from: atherosclerosis, hypertension, IGT (impaired glucose tolerance), diabetes, dyslipidaemia, coronary heart disease, gallbladder disease, osteoarthritis, cancer including endometrial cancer, breast, prostate and colon cancers, bulimia, binge eating, conditions associated with epilepsy, motion sickness, vertigo, dementia, Alzheimer's disease, allergic rhinitis, ulcer, anorexia, migraine hyperactivity disorder, schizophrenia, obesity, ADHD, and cognitive disorders.
